(19) United States
(12) Patent Application Publication
(10) Pub. No.: US 2008/0021067 A1
(43) Pub. Date: Jan. 24, 2008

(54) Methods For The Treatment Of Substance Abuse And Addiction

(76) Inventors: Linda Bristow, Boston, MA (US); Tung M. Fong, Somerset, NJ (US); Andrew C. Morse, San Diego, CA (US)

Correspondence Address: MERCK AND CO., INC, P O BOX 2000, RAHWAY, NJ, 07065-0907, US

(21) Appl. No.: 11/662,018

(22) PCT Filed: Sep. 30, 2005
(86) PCT No.: PCT/US05/35449

§ 371 Date: Mar. 5, 2007

Related U.S. Application Data
(60) Provisional application No. 60/616,064, filed on Oct. 5, 2004.

(51) Int. Cl. A61K 31/454; A61P 25/34

(52) U.S. Cl. 514/326

(57) ABSTRACT

The present invention relates to methods of treating and preventing substance addiction and substance abuse, including nicotine addiction and nicotine addiction-related disorders in a subject comprising administering a melanocortin 4 receptor agonist to said subject. The present invention further relates to methods of treating or preventing substance addiction and substance addiction-related disorders in a subject comprising administering a melanocortin 4 receptor agonist to said subject. The present invention further provides for pharmaceutical compositions and medicaments useful in carrying out these methods.

### BACKGROUND OF THE INVENTION

Substance abuse and substance addiction are public health issues that impose significant social and economic costs on both the addict and on society by playing a major role in violent crime and the transmission of infectious diseases, such as AIDS, hepatitis and tuberculosis. Based on recent figures from the National Institute of Drug Abuse, there are 1.5 million Americans addicted to cocaine, 2.4 million Americans have tried heroin, 4.9 million have tried methamphetamine, 72 million Americans (33% of the population) have tried marijuana, 57 million Americans smoke cigarettes, and 7.6 million use smokeless tobacco.

Nicotine is one of the most widely used addictive drugs and nicotine abuse is the most common form of substance abuse. The World Health Organization (WHO) estimates there are 1.25 billion smokers worldwide, which represents 30% of the global population aged 15 and over. In the industrialized countries of the world, it is estimated that over 170 million people use tobacco. The WHO further estimates that 4 million deaths occur each year as a direct result of tobacco use, which constitutes the largest single preventable cause of death worldwide. As tobacco consumption increases, particularly in young people and in developing countries, it is estimated that it will kill 10 million people by the year 2025. Cigarette smoking is associated with 430,000 deaths a year in the US alone, and is estimated to cost the nation 80 billion dollars a year in health care costs. Tobacco use accounts for one third of all cancers, including cancer of the lung, mouth, pharynx, larynx, esophagus, cervix, kidney, ureter and bladder. The overall rates of death from cancer are twice as high among smokers as among nonsmokers. Smoking also causes lung diseases such as chronic bronchitis, emphysema; exacerbates asthma symptoms; and increases the risk of heart disease, including stroke, heart attack, vascular disease, and aneurysm. An estimated 20% of the deaths from heart disease are attributable to smoking. Women who smoke are at greater risk than nonsmokers for premature delivery, spontaneous abortion, and infants with decreased birth weight.

Nicotine, the primary component in tobacco, is highly addictive. Nicotine use results in increased levels of the neurotransmitter dopamine which activates the reward pathways within the brain circuitry to regulate feelings of pleasure and to mediate the desire to consume nicotine. Chronic exposure to nicotine leads to physical dependence or addiction, accompanied by craving and physical and psychological withdrawal symptoms after the use is reduced or stopped for 24 hours or longer. Withdrawal symptoms, include craving, irritability, anger, hostility, aggression, fatigue, depression, cognitive impairment, such as language comprehension; attention deficits; sleep disturbances; increased appetite; a decrease in social cooperation, and longer periods to regain emotional equilibrium following stress, leading the abuser to seek more nicotine. Repeated use of nicotine also results in the development of tolerance, a condition in which higher doses of nicotine are required to produce the same initial stimulation. Tolerance causes the smoker to continue dosing frequently throughout the day to maintain the drug's pleasurable effects.

Research suggests that people also continue to smoke because of the reinforcing effects of nicotine. Research findings show that when allowed to control the nicotine content of each puff, smokers previously deprived of cigarettes, or administered a centrally acting nicotine antagonist, select higher nicotine concentrations. The rewarding or reinforcing psychopharmacologic effects of nicotine include tranquilization, weight loss, decreased irritability, a reduction in craving for cigarettes and other tobacco products, increased alertness, and improved cognitive function. These effects involve to some extent the relief of withdrawal symptoms, which could be considered negative reinforcement by nicotine.

The addictive nature of nicotine and the unpleasant withdrawal symptoms, which can be relieved by further nicotine use, result in a high failure rate of those trying to quit. Withdrawal symptoms may last a month or more. In particular, craving, or the urge for nicotine, which may persist for 6 months or longer, is described as a major obstacle in successful abstinence. Greater than 90% of those smokers who try to quit without seeking treatment fail, with most relapsing within a week.

Nicotine addiction and abuse is currently treated using nicotine replacement products, such as the nicotine patch (Nicoderm®, Nicotderm CQ®), nicotine chewing gum (Nicorette®), nicotine nasal spray and nicotine inhaler. Nicotine replacement therapies reduce the physiological effects of withdrawal, however, cravings for nicotine often persist. Nicotine replacement products, which provide users with a lower overall nicotine level and produce less severe withdrawal symptoms, have low success rates with only 12-18% of patients successfully having stopped smoking after 52 weeks of treatment. Nicotine addiction and abuse is also treated using non-nicotine replacement products, such as Zyban® (Bupropion), an anti-depressant that has been shown to reduce the withdrawal symptoms and cravings associated with nicotine addiction by blocking neuronal uptake of norepinephrine and dopamine. Zyban has improved efficacy over nicotine replacement therapy with between 23-30% of patients having stopped smoking after 52 weeks of treatment. Other drugs currently used in the treatment of smoking cessation and the prevention of nicotine withdrawal symptoms include anti-depressants such as: Doxepin (Merck Index No. 3425:539); Imipramine (Nunn-Thompson et al., 1989 Clin. Pharm. 8:710-720); Desipramine (Diana et al., 1990 Am. J. Physiol. 259: H1718-H1729); and Nortriptyline®; and the anti-hypertensive Clonidine®. A combination therapy of naltrexone and nicotine for smoking cessation also has been proposed.

Opiate, cocaine, amphetamine and marijuana addiction constitute other major forms of substance addiction. Opiates primarily function as strong analgesics, and are prescribed to treat pain, however, heroin use is often recreational. Opiate use can result in tolerance and dependence, which is characterized by nausea, vomiting, constipation, anorexia, and signs of CNS hyperactivity. Tolerance and physical dependence develop rapidly; short term use of 2-3 days of therapeutic doses results in mild, flu like withdrawal symptoms, while long term use can result in more severe withdrawal symptoms. Physical dependence on opioids necessitates continued use of the same opioid or a related one to prevent withdrawal. Acute intoxication (overdose) is characterized by drowsiness, decreased respiratory rate and depth, hypotension, bradycardia and decreased body temperature. Overall, at least 25% of opiate abusers die within 10-20 years from suicide, homicide, or an infectious disease, such as HIV and hepatitis. Current therapies involve withdrawing the patient from the opiate with methadone, or preventing the action of opiates on the body with naltrexone. Cocaine is a strong central nervous system stimulant that interferes with the reabsorption process of dopamine. The acute use of cocaine can result in pyrexia, hypertension, and cardiac arrhythmias, and chronic use of cocaine can lead to paranoia, audiovisual hallucinations, and addiction. Treatment of cocaine addiction with Desipramine, Amantadine and Bromocriptine has been shown to minimize the symptoms of cocaine withdrawal in preliminary studies (Hall et al., 1990 Pharmacotherapy 10: 47-65; Kosten et al., 1991 NIDA Res. Monogr. 105: 510-511). Amphetamine abuse and addiction produces psychological dependence similar to those produced by cocaine. Although no physical withdrawal symptoms occur, EEG changes occur and the abrupt discontinuance can result in mental depression, intense fatigue and sleepiness. Tolerance occurs over a prolonged period of acute use and may be accompanied by tachycardia; hallucinations; delusions, excitation; anti-social behavior; anxiety reactions; paranoid psychosis with confusion, and memory loss; and can precipitate a schizophrenic episode. Current treatments for amphetamine abuse and addiction include phenothiazines, haloperidol, and chlorpromazine for hallucinations; however the potential side effects of these treatments include postural hypotension, and potentially severe extrapyramidal motor reactions. Marijuana abuse and addiction is primarily psychological. Short term effects of marijuana use can include memory and learning problems; distorted perception; difficulty in thinking and problem solving; loss of coordination; and increased heart rate. Long term use can result in changes in the brain similar to those seen after long-term use of other drugs of abuse, and the same respiratory problems that tobacco smokers have, such as daily cough, phlegm production, frequent acute chest illness, increased risk of lung infections; as well as an increased likelihood of developing cancer of the head, neck and lungs. Depression, anxiety and personality disturbances and job-related “withdrawal behaviors”, such as leaving work without permission, daydreaming, and shirking tasks, have been associated with marijuana use. Long term marijuana use results in addiction in some people, which leads to compulsive use that interferes with family, school, work and recreational activities. Drug craving and withdrawal symptoms, such as irritability, increased aggression, sleeplessness, and anxiety, make it difficult for long-term marijuana users to stop using the drug. There are no medications currently available for treating marijuana abuse, addiction, and relapse.

Although several studies suggest that the melanocortin (MC) peptides, ACTH and α-MSH, play a role in opiate dependence, and that the melanocortin system may modulate drug dependence, the melanocortin receptor subtypes involved are not clear. The involvement of the melanocortin system is best described for the opiate system where the melanocortin agonists (α MSH and ACTH) have been reported to attenuate heroin self administration and to inhibit the development of both tolerance and physical dependence to opiates (Szekely et al., Life Sci. 1979, 24, 1931-38; Contreras & Takemori, J. Phamacol. Exp. Ther., 1984, 229, 21-26). These observations have led to the suggestion that exogenously administered opiates must first overcome an endogenous inhibition by the melanocortin peptides for tolerance and dependence to develop. Consistent with this suggestion, chronic morphine administered in a paradigm known to produce tolerance/dependence led to decreased POMC (propiomelanocortin) mRNA levels in the hypothalamus and decreased melanocortin 4 receptor mRNA in the striatum and accumbens (Alvaro et al., Mol. Pharm., 1996, 50, 583-591). However, dosing protocols producing behavioral sensitization to opiates have been shown to exert the opposite effect and increase striatal and nucleas accumbens MC4 receptor mRNA (Alvaro et al., Life Sci, 1997, 61, 1-9). Several functional studies to date have demonstrated that melanocortins reduce the analgesic effect of morphine (Contreras et al., J. Pharmacol Exp Ther, 229, 21-26 (1984)), however reports also exist to the contrary (Walker et al., Science, 210, 1247-49 (1980); Walker et al., Eur J Pharmacol, 69, 71-79 (1981)). Studies have also shown that similar effects on MC-receptors could be seen after chronic administration of cocaine (Alvaro et al., Life Sci, 1997, 61, 1-9).

The mechanisms through which the melanocortin peptides antagonize the effects of exogenous opiates are unknown. It has been suggested that both MSH-receptor stimulation and MSH-receptor blockade could be effective in treating addiction to morphine and other addictive states, such as cocaine, amphetamine, and narcotic addition. However, it is unclear which, if any, melanocortin receptor subtypes are involved in the effect of the melanocortin peptides (ACTH and a-MSH) on the development of addiction to and tolerance of opiates. Although the MC peptides ACTH and α MSH are known to bind to the melanocortin 4 receptor, these peptides also bind to other melanocortin receptors. α-MSH binds to the melanocortin 1, 4 and 5 receptors; ACTH binds to the melanocortin 1, 2, 3, 4 and 5 receptors. Furthermore, ACTH-like peptides have been reported to bind with micromolar affinity to opiate receptors and to act as partial antagonists. In particular, MSH and ACTH may be very low affinity opiate receptor antagonists (Terenius et al., J. Pharmacol. 33, 395-99 (1975); Gispen et al., Euro J. Pharmacol. 39, 393-7 (1976)). It has also been hypothesized that infusion of MC4-R specific agonists may be expected to block the development of the opiate and cocaine addicted state and to increase the severity of withdrawal. However, it is not clear whether the down-regulation of the melanocortin peptides and the melanocortin 4 receptor by repeated opiate treatment directly contributes to the development of addiction and the expression of withdrawal (Alvaro et al., Life Sci, 1997, 61, 1-9). Nor is it clear that the melanocortin 4 receptor plays a role in the development of addiction. Additionally, several patent applications have disclosed the utility of melanocortin 4 receptor antagonists to treat addictive disorders (WO 00/14115, US 2003/0017966, and WO 02/080758). However, the utility of melanocortin 4 receptor agonists in the treatment of addiction, in particular smoking cessation, and the effects of selective melanocortin 4 agonists on relapse of drug seeking behavior have not been studied to date. The relevance of the melanocortin 4 receptor and the utility of melanocortin 4 agonists to treat addiction and addiction related disorders has not been established.

Despite recent advances, there is a continuing need for new and improved methods of treating and preventing substance abuse, substance addiction and substance addiction related disorders, including seeking behavior, craving and withdrawal symptoms. In particular, there is a need for new and improved methods of treating and preventing nicotine addiction and nicotine addiction related disorders.

It has now been surprisingly found that melanocortin 4 receptor agonists are useful to treat and prevent nicotine addiction. In particular, it has now been discovered that selective melanocortin 4 agonists are useful to reduce nicotine self-administration, and to block the reinforcing effects of nicotine. The use of selective melanocortin 4 receptor agonists is beneficial over the use of non-selective melanocortin agonists since selective MC4R agonists do not exhibit the side effects associated with non-selective MC4R agonists, such as melanocortin 1 mediated pigmentation changes and worsening of acne associated with MC5R agonists. Additionally, the use of non-peptidyl melanocortin-4 receptor agonists for the treatment of nicotine addiction and nicotine addiction related disorders is beneficial over the use of peptidyl melanocortin 4 agonists, which exhibit decreased oral bioavailability due to their degradation in the stomach and GI tract, and which exhibit decreased brain penetration relative to non-peptidyl melanocortin 4 agonists. However, both peptidyl and non-peptidyl melanocortin 4 agonists may be used in the methods of the present invention.

It is an object of the present invention to identify methods of treating and preventing nicotine addiction and nicotine addiction-related disorders comprising administering a melanocortin 4 receptor agonist to a subject. It is another object of the present invention to identify methods of reducing nicotine consumption comprising administering a melanocortin 4 receptor agonist to a subject. It is another object of the present invention to identify methods of blocking the reinforcing effects of nicotine comprising administering a melanocortin 4 receptor agonist to a subject. It is yet another object of the invention to identify methods of treating and preventing substance addiction and substance addiction-related disorders. It is a further object of the present invention to provide a method of manufacture of a medicament useful to treat and prevent substance addiction, including nicotine addiction.

### SUMMARY OF THE INVENTION

The present invention provides a method of treating or preventing nicotine addiction comprising administration of a therapeutically effective amount of a melanocortin 4 receptor agonist to a subject.

The present invention further provides a method of blocking the reinforcing effects of nicotine comprising administration of a therapeutically effective amount of a melanocortin 4 receptor agonist to a subject.

The present invention further provides a method of treating or preventing nicotine abuse comprising administration of a therapeutically effective amount of a melanocortin 4 receptor agonist to a subject.

The present invention further provides a method of treating or preventing nicotine addiction related disorders comprising administration of a therapeutically effective amount of a melanocortin 4 receptor agonist to a subject.

The present invention further provides a method of reducing nicotine consumption comprising administration of a therapeutically effective amount of a melanocortin 4 receptor agonist to a subject.

The present invention further provides a method of reducing nicotine self administration comprising administration of a therapeutically effective amount of a melanocortin 4 receptor agonist to a subject.

The present invention provides a method of treating or preventing substance addiction comprising administration of a therapeutically effective amount of a melanocortin 4 receptor agonist to a subject. The present invention further provides a method of treating or preventing substance abuse comprising administration of a therapeutically effective amount of a melanocortin 4 receptor agonist to a subject. The present invention further provides a method of treating or preventing substance addiction-related disorders comprising administration of a therapeutically effective amount of a melanocortin 4 receptor agonist to a subject. The present invention further provides a method of reducing substance consumption comprising administration of a therapeutically effective amount of a melanocortin 4 receptor agonist to a subject. The present invention further provides a method of inhibiting substance consumption comprising administration of a therapeutically effective amount of a melanocortin 4 receptor agonist to a subject.

The present invention is also concerned with treatment of these conditions, and the use of the compounds and compositions of the present invention for manufacture of a medicament useful for treating these conditions.

### BRIEF DESCRIPTION OF THE DRAWINGS

FIG. 1. Animal Model of Nicotine Reward illustrates the effect of Compound A on maintenance levels of nicotine self-administration. The Baseline data shows the mean number of nicotine infusions for baseline and treatment groups prior to the administration of a MC4R agonist. Melanocortin 4 receptor agonist, Compound A, was administered at 0, or 20 mg/kg, po prior to testing. The Test data shows a significant reduction in nicotine self-administration (nicotine infusions) was observed following administration of 20 mg/kg of Compound A.

FIG. 2. Micro-analysis of nicotine self-administration behavior. Illustrates the effect of Compound A on maintenance levels of nicotine self-administration during the test period. Solid circles indicate lever press and open circles indicate nicotine infusion over the 60 minute test period. Melanocortin 4 receptor agonist, Compound A, was administered at 0, or 20 mg/kg, po prior to testing. A significant reduction in nicotine self-administration was observed following administration of 20 mg/kg of Compound A.

### DETAILED DESCRIPTION OF THE INVENTION

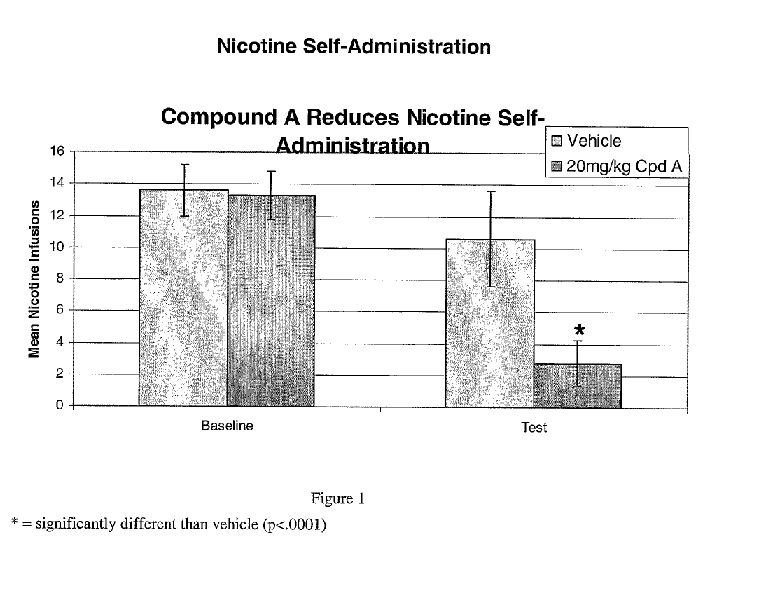

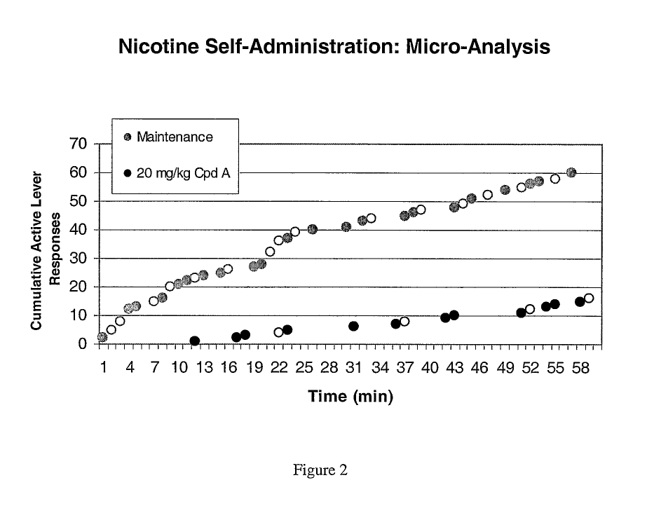

The present invention shows that selective melanocortin 4 receptor agonists inhibit nicotine self-administration and block the reinforcing effects of nicotine. It was found that pre-treatment of Wistar rats with a selective melanocortin-4 receptor agonist, Compound A, reduced nicotine self administration by approximately 85% during a maintenance period of regular nicotine use (See FIGS. 1 and 2). This study showed that a selective melanocortin 4 agonist can block the rewarding effects of nicotine during a period of regular nicotine use. This study also showed that melanocortin 4 receptor agonists block the acute reinforcing effects of nicotine during a period of regular/active nicotine use. Additionally, MC4R agonist Compound A had no significant effects on an alternative lever (non-drug reinforced) suggesting its effects on consumption are specific to nicotine and not a general decrease in activity.

The present invention provides a method of inhibiting or reducing nicotine self administration. The present invention further provides a method of reducing nicotine consumption. The present invention further provides a method of treating or preventing nicotine addiction. The present invention also provides a method of treating or preventing nicotine addiction-related disorders. The present invention further provides a significant reduction in voluntary nicotine consumption. The present invention further provides a method of blocking the reinforcing effects of nicotine. The present invention further provides a method of reducing nicotine craving and withdrawal symptoms. The present invention further relates to a method of treating or preventing substance addiction and substance addiction related disorders. The present invention provides a method of inhibiting substance self administration and consumption. The present invention further provides a method of reducing substance craving and withdrawal symptoms. The present invention also relates to pharmaceutical compositions, and medicaments useful for carrying out these methods.

The methods of the present invention comprise a melanocortin 4 receptor agonist. The melanocortin 4 receptor agonist of use in the present invention is selected from any melanocortin 4 receptor agonist known in the art. Additionally, the melanocortin 4 receptor agonist may be a selective melanocortin 4 agonist. For convenience, the use of an orally active selective melanocortin 4 receptor agonist is preferred. To facilitate dosing, it is preferred that the melanocortin 4 receptor agonist is a long acting melanocortin 4 receptor agonist.

In one embodiment of the present invention, the invention is directed to a method of inhibiting nicotine consumption comprising administering a therapeutically effective amount of a melanocortin 4 receptor agonist to a subject in need thereof.

In another embodiment of the present invention, the invention is directed to a method of reducing nicotine consumption comprising administering a therapeutically effective amount of a melanocortin 4 receptor agonist to a subject in need thereof. In a class of this embodiment, the nicotine consumption is voluntary nicotine consumption.

In another embodiment of the present invention, the invention is directed to a method of blocking the reinforcing effects of nicotine comprising administering a therapeutically effective amount of a melanocortin 4 receptor agonist to a subject in need thereof.

In another embodiment of the present invention, the invention is directed to a method of treating or preventing nicotine addiction comprising administering a therapeutically effective amount of a melanocortin 4 receptor agonist to a subject in need thereof.

In another embodiment of the present invention, the invention is directed to a method of treating or preventing nicotine abuse comprising administering a therapeutically effective amount of a melanocortin 4 receptor agonist to a subject in need thereof.

In another embodiment of the present invention, the invention is directed to a method of treating or preventing nicotine addiction-related disorders comprising administering a therapeutically effective amount of a melanocortin 4 receptor agonist to a subject in need thereof.

In another embodiment of the present invention, the invention is directed to a method of promoting smoking cessation comprising administering a therapeutically effective amount of a melanocortin 4 receptor agonist to a subject in need thereof.

In another embodiment of the present invention, the invention is directed to a method of preventing or reducing the weight gain associated with smoking cessation comprising administering a therapeutically effective amount of a melanocortin 4 receptor agonist to a subject in need thereof.

In another embodiment of the present invention, the invention is directed to a method of preventing relapse of nicotine consumption comprising administering a therapeutically effective amount of a melanocortin 4 receptor agonist to a subject in need thereof.

In another embodiment of the present invention, the invention is directed to a method of increasing the length of abstinence from nicotine consumption comprising administering a therapeutically effective amount of a melanocortin 4 receptor agonist to a subject in need thereof.

In another embodiment of the present invention, the invention is directed to a method of preventing or reducing nicotine craving comprising administering a therapeutically effective amount of a melanocortin 4 receptor agonist to a subject in need thereof.

In another embodiment of the present invention, the invention is directed to a method of preventing or reducing nicotine withdrawal symptoms comprising administering a therapeutically effective amount of a melanocortin 4 receptor agonist to a subject in need thereof.

In another embodiment of the present invention, the invention is directed to a method of decreasing nicotine dependence comprising administering a therapeutically effective amount of a melanocortin 4 receptor agonist to a subject in need thereof.

In another embodiment of the present invention, the invention is directed to a method of preventing a relapse of nicotine consumption to pretreatment levels following a period of nicotine deprivation comprising administering a therapeutically effective amount of a melanocortin 4 receptor agonist to a subject in need thereof.

In another embodiment of the present invention, the invention is directed to a method of reducing the amount of nicotine consumed as the result of relapse of nicotine consumption comprising administering a therapeutically effective amount of a melanocortin 4 receptor agonist to a subject in need thereof.

In another embodiment of the present invention, the invention is directed to a method of reducing nicotine consumption comprising administering to a subject a therapeutically effective amount of a selective melanocortin 4 receptor agonist, or a pharmaceutically acceptable salt thereof, wherein the functional activity of the melanocortin 4 receptor agonist is characterized by an EC₅₀ at least 14-fold more selective for the human melanocortin 4 receptor than for the human melanocortin 1 receptor, human melanocortin 2 receptor, the human melanocortin 3 receptor and the human melanocortin 5 receptor. In another embodiment of the present invention, the invention is directed to a method of reducing nicotine consumption comprising administering to a subject a therapeutically effective amount of a selective melanocortin 4 receptor agonist, or a pharmaceutically acceptable salt thereof, wherein the functional activity of the melanocortin 4 receptor agonist is characterized by an EC₅₀ at least 65-fold more selective for the human melanocortin 4 receptor than for the human melanocortin 1 receptor, human melanocortin 2 receptor, the human melanocortin 3 receptor and the human melanocortin 5 receptor. In another embodiment of the present invention, the invention is directed to a method of reducing nicotine consumption comprising administering to a subject a therapeutically effective amount of a selective melanocortin 4 receptor agonist, or a pharmaceutically acceptable salt thereof, wherein the functional activity of the melanocortin 4 receptor agonist is characterized by an EC₅₀ at least 200-fold more selective for the human melanocortin 4 receptor than for the human melanocortin 1 receptor, human melanocortin 2 receptor, the human melanocortin 3 receptor and the human melanocortin 5 receptor.

In a class of these embodiments, the functional activity of the melanocortin 4 agonist is characterized by an EC₅₀ at least 14-fold more selective for the human melanocortin 4 receptor than for the human melanocortin 1 receptor. In another class of these embodiments, the functional activity of the melanocortin 4 agonist is characterized by an EC₅₀ at least 120-fold more selective for the human melanocortin 4 receptor than for the human melanocortin 1 receptor.

In another class of these embodiments, the functional activity of the melanocortin 4 agonist is characterized by an EC₅₀ at least 100-fold more selective for the human melanocortin 4 receptor than for the human melanocortin 2 receptor. In another class of these embodiments, the functional activity of the melanocortin 4 agonist is characterized by an EC₅₀ at least 700-fold more selective for the human melanocortin 4 receptor than for the human melanocortin 2 receptor.

In another class of these embodiments, the functional activity of the melanocortin 4 agonist is characterized by an EC₅₀ at least 30-fold more selective for the human melanocortin 4 receptor than for the human melanocortin 3 receptor. In another class of these embodiments, the functional activity of the melanocortin 4 agonist is characterized by an EC₅₀ at least 65-fold more selective for the human melanocortin 4 receptor than for the human melanocortin 3 receptor.

In another class of these embodiments, the functional activity of the melanocortin 4 agonist is characterized by an EC₅₀ at least 50-fold more selective for the human melanocortin 4 receptor than for the human melanocortin 5 receptor. In another class of these embodiments, the functional activity of the melanocortin 4 agonist is characterized by an EC₅₀ at least 90-fold more selective for the human melanocortin 4 receptor than for the human melanocortin 5 receptor.

In another embodiment of the present invention, the invention is directed to a method of reducing nicotine consumption comprising administering to a subject a therapeutically effective amount of a melanocortin 4 receptor agonist, or a pharmaceutically acceptable salt thereof, wherein the binding affinity index of the melanocortin 4 receptor agonist is characterized by an IC₅₀ value at least 35-fold more selective for the human melanocortin 4 receptor than for the human melanocortin 1 receptor, the human melanocortin 3 receptor and the human melanocortin 5 receptor.

In a class of this embodiment, the binding affinity index of the melanocortin 4 agonist is characterized by an IC₅₀ value at least 125-fold more selective for the human melanocortin 4 receptor than for the human melanocortin 1 receptor.

In another class of this embodiment, the binding affinity index of the melanocortin 4 agonist is characterized by an IC₅₀ value at least 35-fold more selective for the human melanocortin 4 receptor than for the human melanocortin 3 receptor. In another class of this embodiment, the binding affinity index of the melanocortin 4 agonist is characterized by an IC₅₀ value at least 160-fold more selective for the human melanocortin 4 receptor than for the human melanocortin 3 receptor.

In another class of this embodiment, the binding affinity index of the melanocortin 4 agonist is characterized by an IC₅₀ value at least 12-fold more selective for the human melanocortin 4 receptor than for the human melanocortin 5 receptor. In another class of this embodiment, the binding affinity index of the melanocortin 4 agonist is characterized by an IC₅₀ value at least 35-fold more selective for the human melanocortin 4 receptor than for the human melanocortin 5 receptor.

In another embodiment of the present invention, the invention is directed to the use of a therapeutically effective amount of a melanocortin 4 receptor agonist for the manufacture of a medicament useful to inhibit nicotine consumption in a subject in need of such treatment. In another embodiment of the present invention, the invention is directed to the use of a therapeutically effective amount of a melanocortin 4 receptor agonist for the manufacture of a medicament useful to reduce nicotine consumption in a subject in need of such treatment. In a class of this embodiment, the nicotine consumption is voluntary nicotine consumption.

In another embodiment of the present invention, the invention is directed to the use of a therapeutically effective amount of a melanocortin 4 receptor agonist for the manufacture of a medicament useful to block the reinforcing effects of nicotine in a subject in need of such treatment. In another embodiment of the present invention, the invention is directed to the use of a therapeutically effective amount of a melanocortin 4 receptor agonist for the manufacture of a medicament useful to treat or prevent nicotine addiction in a subject in need of such treatment. In another embodiment of the present invention, the invention is directed to the use of a therapeutically effective amount of a melanocortin 4 receptor agonist for the manufacture of a medicament useful to treat or prevent nicotine abuse in a subject in need of such treatment. In another embodiment of the present invention, the invention is directed to the use of a therapeutically effective amount of a melanocortin 4 receptor agonist for the manufacture of a medicament useful to treat or prevent nicotine addiction-related disorders in a subject in need of such treatment.

In another embodiment of the present invention, the invention is directed to the use of a therapeutically effective amount of a melanocortin 4 receptor agonist for the manufacture of a medicament useful to facilitate smoking cessation in a subject in need of such treatment. In another embodiment of the present invention, the invention is directed to the use of a therapeutically effective amount of a melanocortin 4 receptor agonist for the manufacture of a medicament useful to prevent or reduce the weight gain associated with smoking cessation in a subject in need of such treatment.

In another embodiment of the present invention, the invention is directed to the use of a therapeutically effective amount of a melanocortin 4 receptor agonist for the manufacture of a medicament useful to prevent relapse of nicotine consumption in a subject in need of such treatment. In another embodiment of the present invention, the invention is directed to the use of a therapeutically effective amount of a melanocortin 4 receptor agonist for the manufacture of a medicament useful to increase the length of abstinence from nicotine consumption in a subject in need of such treatment. In another embodiment of the present invention, the invention is directed to the use of a therapeutically effective amount of a melanocortin 4 receptor agonist for the manufacture of a medicament useful to prevent or reduce nicotine craving in a subject in need of such treatment. In another embodiment of the present invention, the invention is directed to the use of a therapeutically effective amount of a melanocortin 4 receptor agonist for the manufacture of a medicament useful to reduce or relieve nicotine withdrawal symptoms in a subject in need of such treatment. In another embodiment of the present invention, the invention is directed to the use of a therapeutically effective amount of a melanocortin 4 receptor agonist for the manufacture of a medicament useful to decrease nicotine dependence in a subject in need of such treatment.

In another embodiment of the present invention, the invention is directed to the use of a therapeutically effective amount of a melanocortin 4 receptor agonist for the manufacture of a medicament useful to prevent a relapse of nicotine consumption to pre-treatment levels following a period of nicotine deprivation or abstinence in a subject in need of such treatment. In another embodiment of the present invention, the invention is directed to the use of a therapeutically effective amount of a melanocortin 4 receptor agonist for the manufacture of a medicament useful to reduce the amount of nicotine consumed as the result of relapse of nicotine consumption in a subject in need of such treatment.

In another embodiment of the present invention, the invention is directed to a method of treating or preventing substance addiction comprising administering a therapeutically effective amount of a melanocortin 4 receptor agonist to a subject in need thereof. In one class of this embodiment, substance addiction is addiction to cocaine, opiates, amphetamines and marijuana.

In another class of this embodiment, substance addiction is addiction to cocaine, and opiates. In yet another class of this embodiment, substance addiction is addiction to amphetamines and marijuana.

In addition to the use of melanocortin 4 agonists to treat nicotine addiction and nicotine addiction related disorders, melanocortin 4 agonists may also be used to treat substance addiction, substance abuse and substance addiction-related disorders. In another embodiment of the present invention, the invention is directed to a method of treating or preventing substance addiction-related disorders comprising administering a therapeutically effective amount of a melanocortin 4 receptor agonist to a subject in need thereof. In one class of this embodiment, substance addiction is addiction to cocaine, opiates, amphetamines and marijuana. In another class of this embodiment, substance addiction is addiction to cocaine, and opiates. In yet another class of this embodiment, substance addiction is addiction to amphetamines and marijuana. In another embodiment of the present invention, the invention is directed to a method of treating or preventing substance addiction comprising administering a therapeutically effective amount of a melanocortin 4 receptor agonist to a subject in need thereof. In one class of this embodiment, substance addiction is addiction to cocaine, opiates, amphetamines and marijuana. In another class of this embodiment, substance addiction is addiction to cocaine, and opiates. In yet another class of this embodiment, substance addiction is addiction to amphetamines and marijuana.

It is further contemplated that melanocortin 4 receptor agonists, particularly selective melanocortin 4 agonists, are useful to treat subjects addicted to other substances, including but not limited to, cocaine addiction, marijuana addiction, opiate addiction, and amphetamine addiction. It is also contemplated that melanocortin 4 receptor agonists, in particular selective melanocortin 4 agonists, are useful for the prevention or reduction of withdrawal symptoms and craving related to substance addiction, including but not limited to, withdrawal symptoms and craving associated with cocaine addiction, opiate addiction, marijuana addiction and amphetamine addiction. It is also contemplated that melanocortin 4 receptor agonists, in particular selective melanocortin 4 agonists, are useful for the prevention or reduction of drug use and drug seeking behavior related to substance addiction, including but not limited to, drug use and drug seeking behavior associated with cocaine addiction, opiate addiction, marijuana addiction and amphetamine addiction.

Melanocortin 4 receptor agonists useful in the methods and medicaments of the present invention are represented by the compounds of structural Formula I and II: 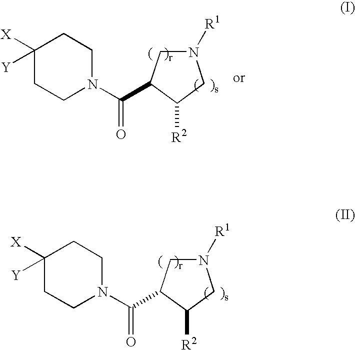
or a pharmaceutically acceptable salt thereof; wherein
X is selected from the group consisting of: C₁₋₈ alkyl, (CH₂)ₙC₃₋₈ cycloalkyl, (CH₂)ₙ-phenyl, (CH₂)ₙ-naphthyl, (CH₂)ₙ-heteroaryl, (CH₂)ₙheterocyclyl, (CH₂)ₙC≡N, (CH₂)ₙCON(R⁵R⁵), (CH₂)ₙCO₂R⁵, (CH₂)ₙCOR⁵, (CH₂)ₙNR⁵C(O)R⁵, (CH₂)ₙNR⁵CO₂R⁵, (CH₂)ₙNR⁵C(O)N(R⁵)₂, (CH₂)ₙNR⁵SO₂R⁵, (CH₂)ₙS(O)ₚR⁵, (CH₂)ₙSO₂N(R⁵)(R⁵), (CH₂)ₙOR⁵, (CH₂)ₙOC(O)R⁵, (CH₂)ₙOC(O)OR⁵, (CH₂)ₙOC(O)N(R⁵)₂, (CH₂)ₙN(R⁵)(R⁵), and (CH₂)ₙNR⁵SO₂N(R⁵)(R⁵); phenyl, naphthyl, and heteroaryl are unsubstituted or substituted with one to three groups independently selected from R⁶; alkyl, cycloalkyl, and heterocyclyl are unsubstituted or substituted with one to three groups independently selected from R⁶ and oxo; and wherein any methylene (CH₂) in X is unsubstituted or substituted with one to two groups independently selected from halogen, hydroxy, and C₁₋₄ alkyl;
Y is selected from the group consisting of: hydrogen, C₁₋₈ alkyl, C₂₋₆ alkenyl, (CH₂)ₙC₃₋₈ cycloalkyl, (CH₂)ₙ-phenyl, (CH₂)ₙ-naphthyl, (CH₂)ₙ-heteroaryl, and (CH₂)ₙ-heterocyclyl; and phenyl, naphthyl, and heteroaryl are unsubstituted or substituted with one to three groups independently selected from R⁶; alkyl, cycloalkyl, and heterocyclyl are optionally substituted with one to three groups independently selected from R⁶ and oxo; and wherein any methylene (CH₂) in Y is unsubstituted or substituted with one to two groups independently selected from halogen, hydroxy, and C₁₋₄ alkyl;
R¹ is selected from the group consisting of: hydrogen, amidino, C₁₋₄ alkyliminoyl, C₁₋₁₀ alkyl, (CH₂)ₙ—C₃₋₇ cycloalkyl, (CH₂)ₙ-phenyl, (CH₂)ₙ-naphthyl, and (CH₂)ₙ-heteroaryl, wherein heteroaryl is selected from the group consisting of (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³; and alkyl and cycloalkyl are unsubstituted or substituted with one to three groups independently selected from R³ and oxo;
R² is selected from the group consisting of: phenyl, naphthyl, and heteroaryl, wherein heteroaryl is selected from the group consisting of: (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³;
each R³ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
each R⁴ is independently selected from the group consisting of: hydrogen, C₁₋₆ alkyl, (CH₂)ₙ phenyl, (CH₂)ₙ-heteroaryl, (CH₂)ₙ-naphthyl, (CH₂)ₙ-heterocyclyl, (CH₂)ₙC₃₋₇ cycloalkyl, and (CH₂)ₙC₃₋₇ bicycloalkyl, wherein alkyl, phenyl, heteroaryl, heterocyclyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl;
each R⁵ is independently selected from the group consisting of: hydrogen, C₁₋₈ alkyl, (CH₂)ₙ-phenyl, (CH₂)ₙ-naphthyl, (CH₂)ₙ-heteroaryl, and (CH₂)ₙC₃₋₇ cycloalkyl; phenyl, naphthyl, and heteroaryl are unsubstituted or substituted with one to three groups independently selected from R³; alkyl and cycloalkyl are unsubstituted or substituted with one to three groups independently selected from R³ and oxo; and wherein any methylene (CH₂) in R⁵ is unsubstituted or substituted with one to two groups independently selected from halogen, hydroxy, and C₁₋₄ alkyl; or two R⁵ groups together with the atom to which they are attached form a 5- to 8-membered mono- or bicyclic ring system optionally containing an additional heteroatom selected from O, S, and NC₁₋₄ alkyl;
each R⁶ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R⁶ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
r is 1 or 2;
s is 0, 1, or 2;
n is 0, 1 or 2; and
p is 0, 1, or 2.

In one embodiment of the compounds of structural formula I and II, R¹ is selected from the group consisting of hydrogen, C₁₋₆ alkyl, (CH₂)₀₋₁C₃₋₆ cycloalkyl, and (CH₂)₀₋₁-phenyl; wherein phenyl is unsubstituted or substituted with one to three groups independently selected from R³; and alkyl and cycloalkyl are optionally substituted with one to three groups independently selected from R³ and oxo. In a class of this embodiment, R¹ is tert-butyl.

In a second embodiment of the compounds of structural formula I and II, R² is phenyl or thienyl optionally substituted with one to three groups independently selected from R³. In a class of this embodiment, R² is phenyl optionally substituted with one to three groups independently selected from R³. In another class of this embodiment, R² is phenyl substituted with one to three groups independently selected from R³. In a subclass of this class, R² is phenyl substituted with two groups independently selected from R³. In a subclass of this subclass, R² is phenyl substituted with two halogen groups.

In a third embodiment of the compounds of structural formula I and II, X is selected from the group consisting of: (CH₂)ₙ-phenyl, (CH₂)ₙ-naphthyl, (CH₂)ₙ-heteroaryl, (CH₂)ₙC₃₋₈ cycloalkyl, and (CH₂)ₙ-heterocyclyl; and phenyl, naphthyl, and heteroaryl are optionally substituted with one to three groups independently selected from R⁶; cycloalkyl and heterocyclyl are optionally substituted with one to three groups independently selected from R⁶ and oxo; and wherein any methylene (CH₂) group in X is unsubstituted or substituted with one to two groups independently selected from halogen, hydroxy, and C₁₋₄ alkyl. In a class of this embodiment, X is selected from the group consisting of (CH₂)₀₋₁-phenyl, (CH₂)₀₋₁-heteroaryl, (CH₂)₀₋₁-heterocyclyl; wherein phenyl and heteroaryl are optionally substituted with one to three groups independently selected from R⁶; heterocyclyl is optionally substituted with one to three groups independently selected from R⁶ and oxo; and CH₂ is unsubstituted or substituted with one to two groups independently selected from halogen, hydroxy, and C₁₋₄ alkyl. In a subclass of this class, X is phenyl optionally substituted with one to three groups independently selected from R⁶.

In a fourth embodiment of compounds of formula I and II, Y is hydrogen.

In yet a further embodiment of compounds of structural formula I and II, r is 1 or 2 and s is 1.

In another embodiment of the present invention, the invention comprises a method of treating substance abuse or addiction in a subject in need thereof comprising administering a therapeutically effective amount of a selective melanocortin 4 receptor agonist. In a class of this embodiment, the melanocortin 4 receptor agonist is orally active. In another class of this embodiment, substance is selected from the group consisting of: cocaine, an opiate, an amphetamine, and marijuana.

In another embodiment of the present invention, the invention comprises a method of treating nicotine addiction in a subject in need thereof comprising administering a therapeutically effective amount of a melanocortin 4 receptor agonist. In a class of this embodiment, the melanocortin 4 receptor agonist is a selective melanocortin 4 receptor agonist. In another class of this embodiment, the melanocortin 4 receptor agonist is a compound of Formula I or II: 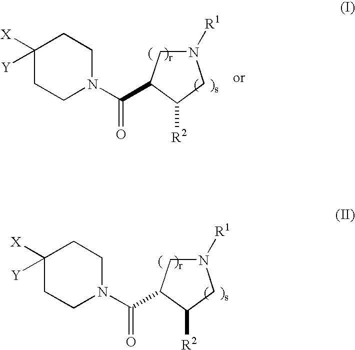
or a pharmaceutically acceptable salt thereof; wherein
X is selected from the group consisting of: C₁₋₈ alkyl, (CH₂)ₙC₃₋₈ cycloalkyl, (CH₂)ₙ-phenyl, (CH₂)ₙ-naphthyl, (CH₂)ₙ-heteroaryl, (CH₂)ₙheterocyclyl, (CH₂)ₙC≡N, (CH₂)ₙCON(R⁵R⁵), (CH₂)ₙCO₂R⁵, (CH₂)ₙCOR⁵, (CH₂)ₙNR⁵C(O)R⁵, (CH₂)ₙNR⁵CO₂R⁵, (CH₂)ₙNR⁵C(O)N(R⁵)₂, (CH₂)ₙNR⁵SO₂R⁵, (CH₂)ₙS(O)ₚR⁵, (CH₂)ₙSO₂N(R⁵)(R⁵), (CH₂)ₙOR⁵, (CH₂)ₙOC(O)R⁵, (CH₂)ₙOC(O)OR⁵, (CH₂)ₙOC(O)N(R⁵)₂, (CH₂)ₙN(R⁵)(R⁵), and (CH₂)ₙNR⁵SO₂N(R⁵)(R⁵); phenyl, naphthyl, and heteroaryl are unsubstituted or substituted with one to three groups independently selected from R⁶; alkyl, cycloalkyl, and heterocyclyl are unsubstituted or substituted with one to three groups independently selected from R⁶ and oxo; and wherein any methylene (CH₂) in X is unsubstituted or substituted with one to two groups independently selected from halogen, hydroxy, and C₁₋₄ alkyl;
Y is selected from the group consisting of: hydrogen, C₁₋₈ alkyl, C₂₋₆ alkenyl, (CH₂)ₙC₃₋₈ cycloalkyl, (CH₂)ₙ-phenyl, (CH₂)ₙ-naphthyl, (CH₂)ₙ-heteroaryl, and (CH₂)ₙ-heterocyclyl; and phenyl, naphthyl, and heteroaryl are unsubstituted or substituted with one to three groups independently selected from R⁶; alkyl, cycloalkyl, and heterocyclyl are optionally substituted with one to three groups independently selected from R⁶ and oxo; and wherein any methylene (CH₂) in Y is unsubstituted or substituted with one to two groups independently selected from halogen, hydroxy, and C₁₋₄ alkyl;
R¹ is selected from the group consisting of: hydrogen, amidino, C₁₋₄ alkyliminoyl, C₁₋₁₀ alkyl, (CH₂)ₙ—C₃₋₇ cycloalkyl, (CH₂)ₙ-phenyl, (CH₂)ₙ-naphthyl, and (CH₂)ₙ-heteroaryl, wherein heteroaryl is selected from the group consisting of (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³; and alkyl and cycloalkyl are unsubstituted or substituted with one to three groups independently selected from R³ and oxo;
R² is selected from the group consisting of: phenyl, naphthyl, and heteroaryl, wherein heteroaryl is selected from the group consisting of: (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³;
each R³ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
each R⁴ is independently selected from the group consisting of: hydrogen, C₁₋₆ alkyl, (CH₂)ₙ phenyl, (CH₂)ₙ-heteroaryl, (CH₂)ₙ-naphthyl, (CH₂)ₙ-heterocyclyl, (CH₂)ₙC₃₋₇ cycloalkyl, and (CH₂)ₙC₃₋₇ bicycloalkyl, wherein alkyl, phenyl, heteroaryl, heterocyclyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl;
each R⁵ is independently selected from the group consisting of: hydrogen, C₁₋₈ alkyl, (CH₂)ₙ-phenyl, (CH₂)ₙ-naphthyl, (CH₂)ₙ-heteroaryl, and (CH₂)ₙC₃₋₇ cycloalkyl; phenyl, naphthyl, and heteroaryl are unsubstituted or substituted with one to three groups independently selected from R³; alkyl and cycloalkyl are unsubstituted or substituted with one to three groups independently selected from R³ and oxo; and wherein any methylene (CH₂) in R⁵ is unsubstituted or substituted with one to two groups independently selected from halogen, hydroxy, and C₁₋₄ alkyl; or two R⁵ groups together with the atom to which they are attached form a 5- to 8-membered mono- or bicyclic ring system optionally containing an additional heteroatom selected from O, S, and NC₁₋₄ alkyl;
each R⁶ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R⁶ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
r is 1 or 2;
s is 0, 1, or 2;
n is 0, 1 or 2; and
p is 0, 1, or 2.
In a subclass of this class, X is phenyl substituted with two groups independently selected from R³, Y is hydrogen, and R¹ is tert-butyl, and R² is phenyl substituted with two groups independently selected from R³, or a pharmaceutically acceptable salt thereof. In another subclass of this class, the melanocortin 4 receptor agonist of Formula I is 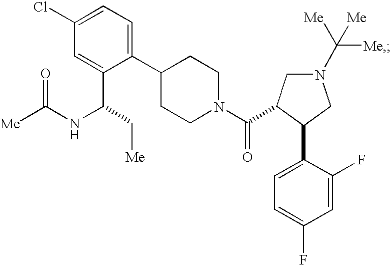
or a pharmaceutically acceptable salt thereof.

In another embodiment of the present invention, the invention comprises a method of facilitating smoking cessation in a subject in need thereof comprising administering a therapeutically effective amount of a melanocortin 4 receptor agonist.

In another embodiment of the present invention, the invention comprises a method of reducing nicotine consumption in a subject in need thereof comprising administering a therapeutically effective amount of a melanocortin 4 receptor agonist. In a class of this embodiment, the melanocortin 4 receptor agonist has a selective functional activity characterized by an EC₅₀ at least 65-fold lower for the human melanocortin 4 receptor than for the human melanocortin 1 receptor, the melanocortin 2 receptor, the human melanocortin 3 receptor and the human melanocortin 5 receptor. In a subclass of this class, the functional activity of the selective melanocortin 4 receptor agonist is characterized by an EC₅₀ at least 120-fold lower for the human melanocortin 4 receptor than for the human melanocortin 1 receptor. In another subclass of this class, the functional activity of the selective melanocortin 4 receptor agonist is characterized by an EC₅₀ at least 700-fold lower for the human melanocortin 4 receptor than for the human melanocortin 2 receptor. In another subclass of this class, the functional activity of the selective melanocortin 4 receptor agonist is characterized by an EC₅₀ at least 90-fold lower for the human melanocortin 4 receptor than for the human melanocortin 5 receptor. In another subclass of this class, the selective melanocortin 4 receptor agonist has a binding affinity index (IC₅₀ value) of less than 45 nM at the human melanocortin 4 receptor.

In another embodiment of the present invention, the invention comprises a method of inhibiting nicotine consumption comprising administering a melanocortin 4 receptor agonist, or a pharmaceutically acceptable salt thereof, to a subject in need thereof wherein the melanocortin 4 receptor agonist is a compound of Formula I or II: 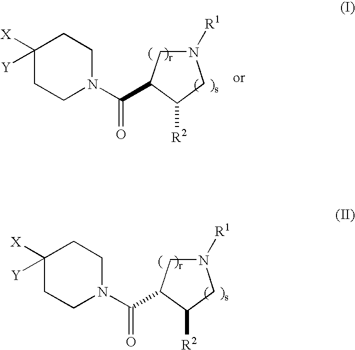
or a pharmaceutically acceptable salt thereof; wherein
X is selected from the group consisting of: C₁₋₈ alkyl, (CH₂)ₙC₃₋₈ cycloalkyl, (CH₂)ₙ-phenyl, (CH₂)ₙ-naphthyl, (CH₂)ₙ-heteroaryl, (CH₂)ₙheterocyclyl, (CH₂)ₙC≡N, (CH₂)ₙCON(R⁵R⁵), (CH₂)ₙCO₂R⁵, (CH₂)ₙCOR⁵, (CH₂)ₙNR⁵C(O)R⁵, (CH₂)ₙNR⁵CO₂R⁵, (CH₂)ₙNR⁵C(O)N(R⁵)₂, (CH₂)ₙNR⁵SO₂R⁵, (CH₂)ₙS(O)ₚR⁵, (CH₂)ₙSO₂N(R⁵)(R⁵), (CH₂)ₙOR⁵, (CH₂)ₙOC(O)R⁵, (CH₂)ₙOC(O)OR⁵, (CH₂)ₙOC(O)N(R⁵)₂, (CH₂)ₙN(R⁵)(R⁵), and (CH₂)ₙNR⁵SO₂N(R⁵)(R⁵); phenyl, naphthyl, and heteroaryl are unsubstituted or substituted with one to three groups independently selected from R⁶; alkyl, cycloalkyl, and heterocyclyl are unsubstituted or substituted with one to three groups independently selected from R⁶ and oxo; and wherein any methylene (CH₂) in X is unsubstituted or substituted with one to two groups independently selected from halogen, hydroxy, and C₁₋₄ alkyl;
Y is selected from the group consisting of: hydrogen, C₁₋₈ alkyl, C₂₋₆ alkenyl, (CH₂)ₙC₃₋₈ cycloalkyl, (CH₂)ₙ-phenyl, (CH₂)ₙ-naphthyl, (CH₂)ₙ-heteroaryl, and (CH₂)ₙ-heterocyclyl; and phenyl, naphthyl, and heteroaryl are unsubstituted or substituted with one to three groups independently selected from R⁶; alkyl, cycloalkyl, and heterocyclyl are optionally substituted with one to three groups independently selected from R⁶ and oxo; and wherein any methylene (CH₂) in Y is unsubstituted or substituted with one to two groups independently selected from halogen, hydroxy, and C₁₋₄ alkyl;
R¹ is selected from the group consisting of: hydrogen, amidino, C₁₋₄ alkyliminoyl, C₁₋₁₀ alkyl, (CH₂)ₙ—C₃₋₇ cycloalkyl, (CH₂)ₙ-phenyl, (CH₂)ₙ-naphthyl, and (CH₂)ₙ-heteroaryl, wherein heteroaryl is selected from the group consisting of (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³; and alkyl and cycloalkyl are unsubstituted or substituted with one to three groups independently selected from R³ and oxo;
R² is selected from the group consisting of: phenyl, naphthyl, and heteroaryl, wherein heteroaryl is selected from the group consisting of: (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³;
each R³ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
each R⁴ is independently selected from the group consisting of: hydrogen, C₁₋₆ alkyl, (CH₂)ₙ phenyl, (CH₂)ₙ-heteroaryl, (CH₂)ₙ-naphthyl, (CH₂)ₙ-heterocyclyl, (CH₂)ₙC₃₋₇ cycloalkyl, and (CH₂)ₙC₃₋₇ bicycloalkyl, wherein alkyl, phenyl, heteroaryl, heterocyclyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl;
each R⁵ is independently selected from the group consisting of: hydrogen, C₁₋₈ alkyl, (CH₂)ₙ-phenyl, (CH₂)ₙ-naphthyl, (CH₂)ₙ-heteroaryl, and (CH₂)ₙC₃₋₇ cycloalkyl; phenyl, naphthyl, and heteroaryl are unsubstituted or substituted with one to three groups independently selected from R³; alkyl and cycloalkyl are unsubstituted or substituted with one to three groups independently selected from R³ and oxo; and wherein any methylene (CH₂) in R⁵ is unsubstituted or substituted with one to two groups independently selected from halogen, hydroxy, and C₁₋₄ alkyl; or two R⁵ groups together with the atom to which they are attached form a 5- to 8-membered mono- or bicyclic ring system optionally containing an additional heteroatom selected from O, S, and NC₁₋₄ alkyl;
each R⁶ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R⁶ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
r is 1 or 2;
s is 0, 1, or 2;
n is 0, 1 or 2; and
p is 0, 1, or 2.

In another embodiment of the present invention, the invention comprises a method of treating nicotine addiction comprising administering a melanocortin 4 receptor agonist, or a pharmaceutically acceptable salt thereof, to a subject in need thereof wherein the melanocortin 4 receptor agonist is a compound of Formula I or II: 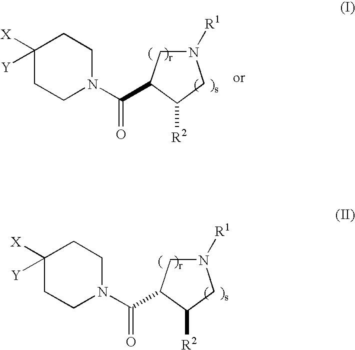
or a pharmaceutically acceptable salt thereof; wherein
X is selected from the group consisting of: C₁₋₈ alkyl, (CH₂)ₙC₃₋₈ cycloalkyl, (CH₂)ₙ-phenyl, (CH₂)ₙ-naphthyl, (CH₂)ₙ-heteroaryl, (CH₂)ₙheterocyclyl, (CH₂)ₙC≡N, (CH₂)ₙCON(R⁵R⁵), (CH₂)ₙCO₂R⁵, (CH₂)ₙCOR⁵, (CH₂)ₙNR⁵C(O)R⁵, (CH₂)ₙNR⁵CO₂R⁵, (CH₂)ₙNR⁵C(O)N(R⁵)₂, (CH₂)ₙNR⁵SO₂R⁵, (CH₂)ₙS(O)ₚR⁵, (CH₂)ₙSO₂N(R⁵)(R⁵), (CH₂)ₙOR⁵, (CH₂)ₙOC(O)R⁵, (CH₂)ₙOC(O)OR⁵, (CH₂)ₙOC(O)N(R⁵)₂, (CH₂)ₙN(R⁵)(R⁵), and (CH₂)ₙNR⁵SO₂N(R⁵)(R⁵); phenyl, naphthyl, and heteroaryl are unsubstituted or substituted with one to three groups independently selected from R⁶; alkyl, cycloalkyl, and heterocyclyl are unsubstituted or substituted with one to three groups independently selected from R⁶ and oxo; and wherein any methylene (CH₂) in X is unsubstituted or substituted with one to two groups independently selected from halogen, hydroxy, and C₁₋₄ alkyl;
Y is selected from the group consisting of: hydrogen, C₁₋₈ alkyl, C₂₋₆ alkenyl, (CH₂)ₙC₃₋₈ cycloalkyl, (CH₂)ₙ-phenyl, (CH₂)ₙ-naphthyl, (CH₂)ₙ-heteroaryl, and (CH₂)ₙ-heterocyclyl; and phenyl, naphthyl, and heteroaryl are unsubstituted or substituted with one to three groups independently selected from R⁶; alkyl, cycloalkyl, and heterocyclyl are optionally substituted with one to three groups independently selected from R⁶ and oxo; and wherein any methylene (CH₂) in Y is unsubstituted or substituted with one to two groups independently selected from halogen, hydroxy, and C₁₋₄ alkyl;
R¹ is selected from the group consisting of: hydrogen, amidino, C₁₋₄ alkyliminoyl, C₁₋₁₀ alkyl, (CH₂)ₙ—C₃₋₇ cycloalkyl, (CH₂)ₙ-phenyl, (CH₂)ₙ-naphthyl, and (CH₂)ₙ-heteroaryl, wherein heteroaryl is selected from the group consisting of (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³; and alkyl and cycloalkyl are unsubstituted or substituted with one to three groups independently selected from R³ and oxo;
R² is selected from the group consisting of: phenyl, naphthyl, and heteroaryl, wherein heteroaryl is selected from the group consisting of: (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and
(21) benzoxazolyl; in which phenyl, naphthyl, and heteroaryl are unsubstituted or substituted with one to three groups independently selected from R³;
each R³ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
each R⁴ is independently selected from the group consisting of: hydrogen, C₁₋₆ alkyl, (CH₂)ₙ phenyl, (CH₂)ₙ-heteroaryl, (CH₂)ₙ-naphthyl, (CH₂)ₙ-heterocyclyl, (CH₂)ₙC₃₋₇ cycloalkyl, and (CH₂)ₙC₃₋₇ bicycloalkyl, wherein alkyl, phenyl, heteroaryl, heterocyclyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl;
each R⁵ is independently selected from the group consisting of: hydrogen, C₁₋₈ alkyl, (CH₂)ₙ-phenyl, (CH₂)ₙ-naphthyl, (CH₂)ₙ-heteroaryl, and (CH₂)ₙC₃₋₇ cycloalkyl; phenyl, naphthyl, and heteroaryl are unsubstituted or substituted with one to three groups independently selected from R³; alkyl and cycloalkyl are unsubstituted or substituted with one to three groups independently selected from R³ and oxo; and wherein any methylene (CH₂) in R⁵ is unsubstituted or substituted with one to two groups independently selected from halogen, hydroxy, and C₁₋₄ alkyl; or two R⁵ groups together with the atom to which they are attached form a 5- to 8-membered mono- or bicyclic ring system optionally containing an additional heteroatom selected from O, S, and NC₁₋₄ alkyl;
each R⁶ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R⁶ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
r is 1 or 2;
s is 0, 1, or 2;
n is 0, 1 or 2; and
p is 0, 1, or 2.

In another embodiment of the present invention, the invention comprises a method of preventing nicotine addiction comprising administering a melanocortin 4 receptor agonist, or a pharmaceutically acceptable salt thereof, to a subject in need thereof wherein the melanocortin 4 receptor agonist is a compound of Formula I or II: 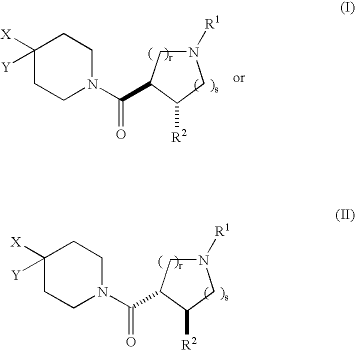
or a pharmaceutically acceptable salt thereof; wherein
X is selected from the group consisting of: C₁₋₈ alkyl, (CH₂)ₙC₃₋₈ cycloalkyl, (CH₂)ₙ-phenyl, (CH₂)ₙ-naphthyl, (CH₂)ₙ-heteroaryl, (CH₂)ₙheterocyclyl, (CH₂)ₙC≡N, (CH₂)ₙCON(R⁵R⁵), (CH₂)ₙCO₂R⁵, (CH₂)ₙCOR⁵, (CH₂)ₙNR⁵C(O)R⁵, (CH₂)ₙNR⁵CO₂R⁵, (CH₂)ₙNR⁵C(O)N(R⁵)₂, (CH₂)ₙNR⁵SO₂R⁵, (CH₂)ₙS(O)ₚR⁵, (CH₂)ₙSO₂N(R⁵)(R⁵), (CH₂)ₙOR⁵, (CH₂)ₙOC(O)R⁵, (CH₂)ₙOC(O)OR⁵, (CH₂)ₙOC(O)N(R⁵)₂, (CH₂)ₙN(R⁵)(R⁵), and (CH₂)ₙNR⁵SO₂N(R⁵)(R⁵); phenyl, naphthyl, and heteroaryl are unsubstituted or substituted with one to three groups independently selected from R⁶; alkyl, cycloalkyl, and heterocyclyl are unsubstituted or substituted with one to three groups independently selected from R⁶ and oxo; and wherein any methylene (CH₂) in X is unsubstituted or substituted with one to two groups independently selected from halogen, hydroxy, and C₁₋₄ alkyl;
Y is selected from the group consisting of: hydrogen, C₁₋₈ alkyl, C₂₋₆ alkenyl, (CH₂)ₙC₃₋₈ cycloalkyl, (CH₂)ₙ-phenyl, (CH₂)ₙ-naphthyl, (CH₂)ₙ-heteroaryl, and (CH₂)ₙ-heterocyclyl; and phenyl, naphthyl, and heteroaryl are unsubstituted or substituted with one to three groups independently selected from R⁶; alkyl, cycloalkyl, and heterocyclyl are optionally substituted with one to three groups independently selected from R⁶ and oxo; and wherein any methylene (CH₂) in Y is unsubstituted or substituted with one to two groups independently selected from halogen, hydroxy, and C₁₋₄ alkyl;
R¹ is selected from the group consisting of: hydrogen, amidino, C₁₋₄ alkyliminoyl, C₁₋₁₀ alkyl, (CH₂)ₙ—C₃₋₇ cycloalkyl, (CH₂)ₙ-phenyl, (CH₂)ₙ-naphthyl, and (CH₂)ₙ-heteroaryl, wherein heteroaryl is selected from the group consisting of (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³; and alkyl and cycloalkyl are unsubstituted or substituted with one to three groups independently selected from R³ and oxo;
R² is selected from the group consisting of: phenyl, naphthyl, and heteroaryl, wherein heteroaryl is selected from the group consisting of: (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and
(21) benzoxazolyl; in which phenyl, naphthyl, and heteroaryl are unsubstituted or substituted with one to three groups independently selected from R³;
each R³ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
each R⁴ is independently selected from the group consisting of: hydrogen, C₁₋₆ alkyl, (CH₂)ₙ phenyl, (CH₂)ₙ-heteroaryl, (CH₂)ₙ-naphthyl, (CH₂)ₙ-heterocyclyl, (CH₂)ₙC₃₋₇ cycloalkyl, and (CH₂)ₙC₃₋₇ bicycloalkyl, wherein alkyl, phenyl, heteroaryl, heterocyclyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl;
each R⁵ is independently selected from the group consisting of: hydrogen, C₁₋₈ alkyl, (CH₂)ₙ-phenyl, (CH₂)ₙ-naphthyl, (CH₂)ₙ-heteroaryl, and (CH₂)ₙC₃₋₇ cycloalkyl; phenyl, naphthyl, and heteroaryl are unsubstituted or substituted with one to three groups independently selected from R³; alkyl and cycloalkyl are unsubstituted or substituted with one to three groups independently selected from R³ and oxo; and wherein any methylene (CH₂) in R⁵ is unsubstituted or substituted with one to two groups independently selected from halogen, hydroxy, and C₁₋₄ alkyl; or two R⁵ groups together with the atom to which they are attached form a 5- to 8-membered mono- or bicyclic ring system optionally containing an additional heteroatom selected from O, S, and NC₁₋₄ alkyl;
each R⁶ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R⁶ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
r is 1 or 2;
s is 0, 1, or 2;
n is 0, 1 or 2; and
p is 0, 1, or 2.

A specific melanocortin-4 receptor agonist useful in the methods of the present invention is Compound A:

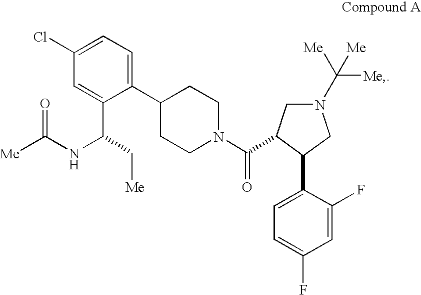

Compound A is a selective agonist for MC4R, with a selective functional activity characterized by an EC₅₀ value for the melanocortin 4 receptor that is at least 120-fold lower than for the melanocortin 1 receptor, at least 700-fold lower than for the melanocortin 2 receptor, at least 65-fold lower than for the melanocortin 3 receptor, and at least 100 fold lower than for the melanocortin 5 receptor.

Other melanocortin-4 receptor agonists useful in the methods of the present invention include, but are not limited to, the following: 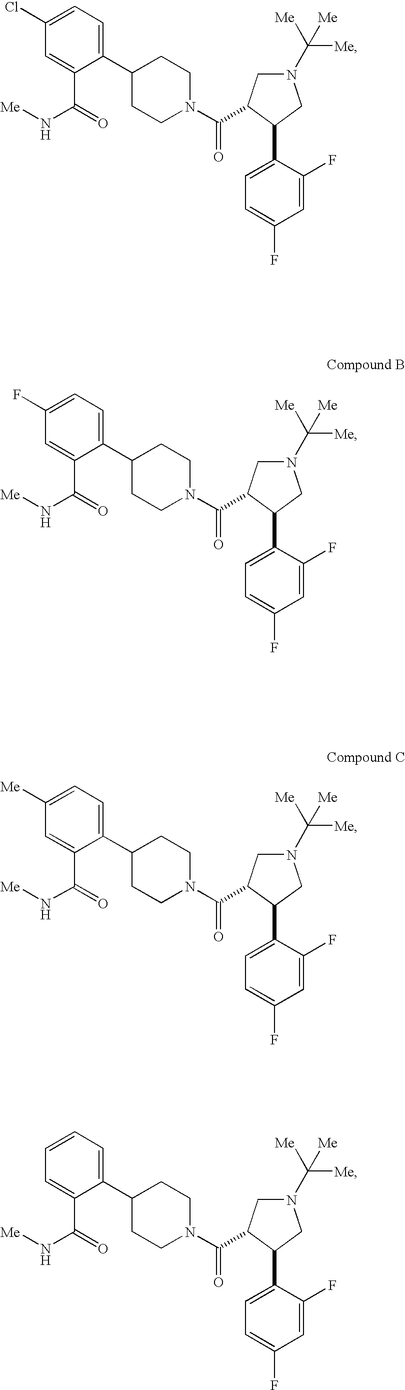 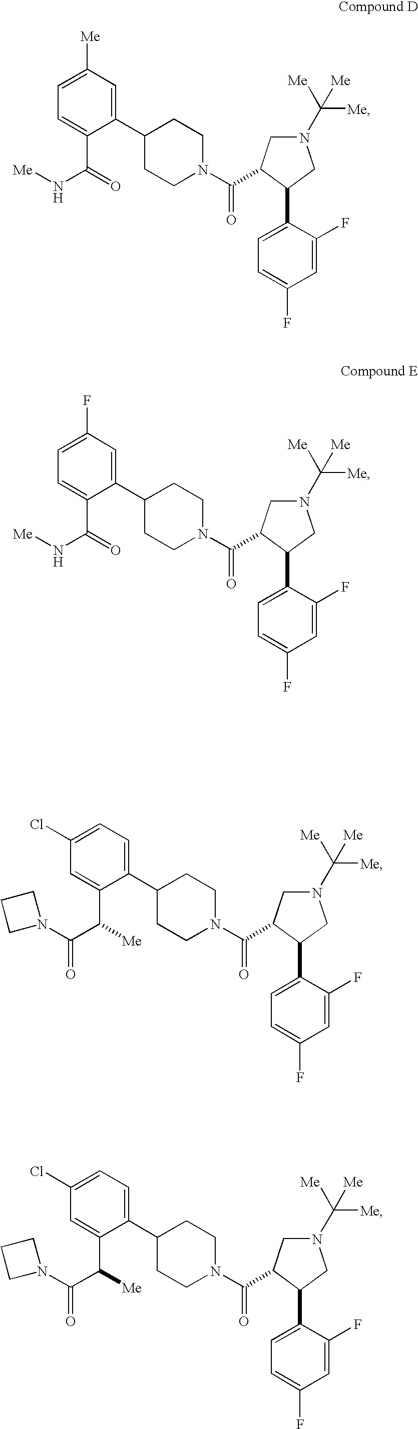 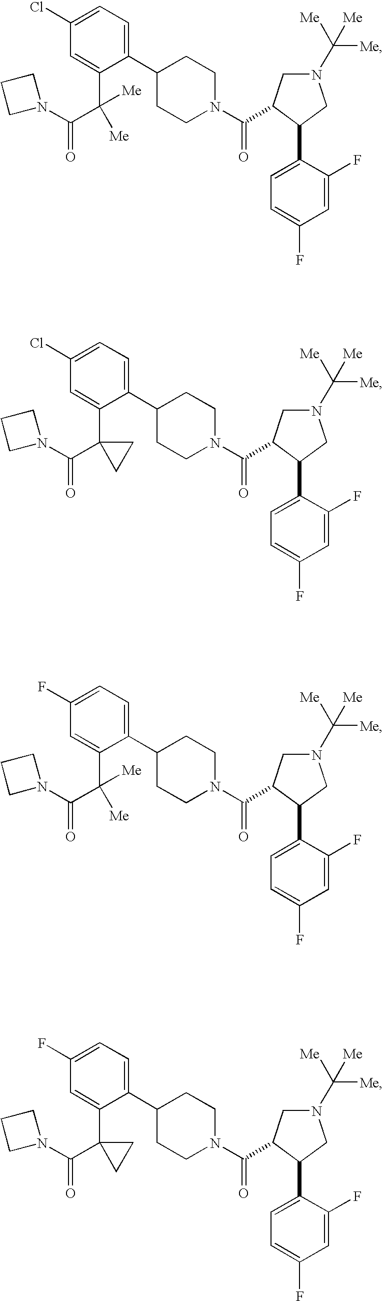 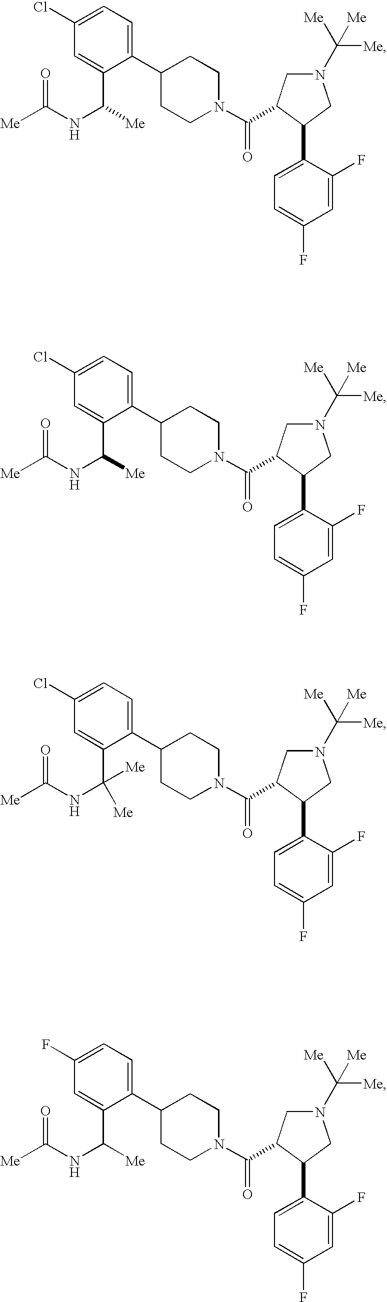 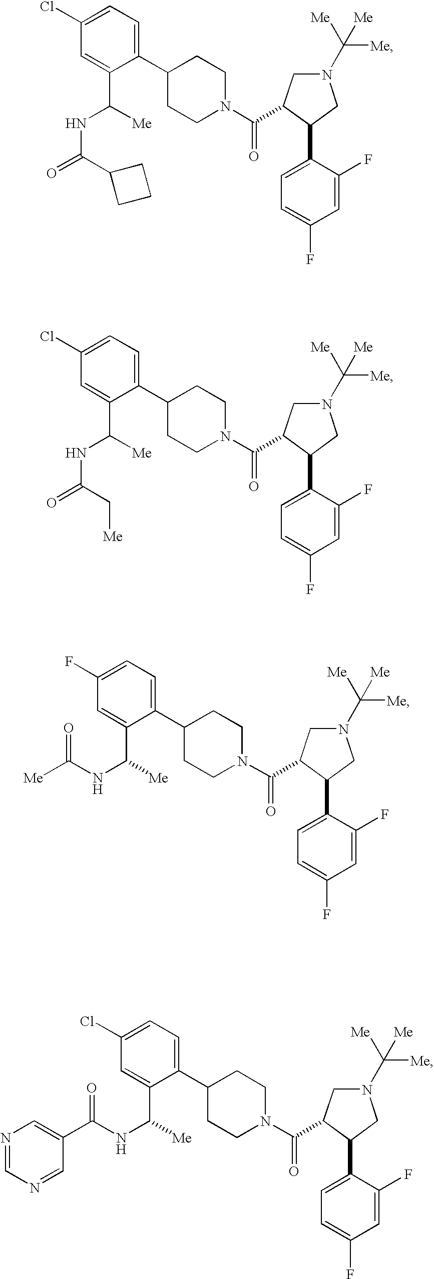 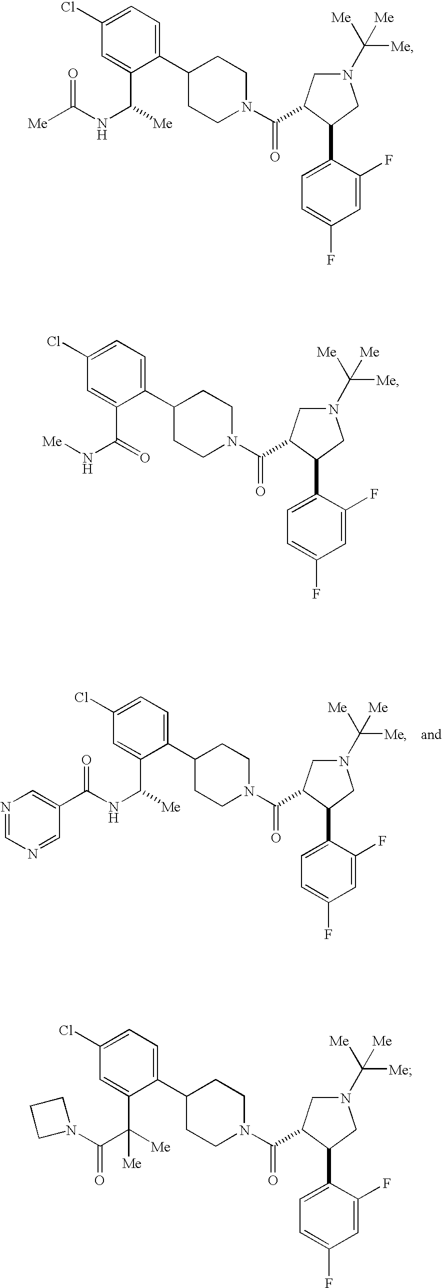
or a pharmaceutically acceptable salt thereof.

The melanocortin 4 receptor agonists of Formula I and II, including Compound A, and their preparation are disclosed in US2003/0225060, which is hereby incorporated by reference in its entirety, WO 02/068387, and WO 02/068388.

The above compounds are only illustrative of the MC4R agonists that can be used in the compositions of the present invention. As this listing of compounds is not meant to be comprehensive, the methods of the present invention may employ any MC4R agonists, including the MC4R agonists of Formulas I, II, and Compound A, and are not limited to any particular structural class of compounds.

Other melanocortin 4 receptor agonists useful in the present invention, include but are not limited to those disclosed in U.S. Pat. No. 6,294,534, U.S. Pat. Nos. 6,350,760, 6,376,509, 6,410,548, 6,458,790, U.S. Pat. No. 6,472,398, U.S. Pat. No. 5,837,521, U.S. Pat. No. 6,699,873, which are hereby incorporated by reference in their entirety; in US Patent Application Publication Nos. US 2002/0004512, US2002/0019523, US2002/0137664, US2003/0236262, US2003/0225060, US2003/0092732, US2003/109556, US 2002/0177151, US 2002/187932, US 2003/0113263, which are hereby incorporated by reference in their entirety; and in WO 99/64002, WO 00/74679, WO 02/15909, WO 01/70708, WO 01/70337, WO 01/91752, WO 02/059095; WO 02/059107; WO 02/059108; WO 02/059117; WO 02/068387, WO 02/068388, WO 02/067869, WO 03/007949, WO 2004/024720, WO 2004/037797, WO 01/58891, WO 02/070511, WO 02/079146, WO 03/009847, WO 03/057671, WO 03/068738, WO 03/092690, WO 02/059095, WO 02/059107, WO 02/059108, WO 02/059117, WO 02/085925, WO 03/004480, WO 03/007949; WO 03/009847; WO 03/009850, WO 03/013571, WO 03/031410, WO 03/053927, WO 03/061660, WO 03/066597, WO 03/094918, WO 03/099818, WO 04/037797, WO 04/048345, WO 04/058735, WO 02/018327, WO 02/080896, WO 02/081443, WO 03/066587, WO 03/066597, WO 03/099818, WO 02/062766, WO 03/000663, WO 03/000666, WO 03/003977, WO 03/040107, WO 03/040117, WO 03/040118, WO 03/013509, WO 03/057671, WO 02/079753, WO 02/092566, WO 03/093234, WO 03/095474; WO 03/104761; WO 04/089307; WO 04/078717; WO 04/087159; and WO 05/009950.

One of ordinary skill in the art can readily identify melanocortin 4 receptor agonist compounds useful in the compositions and methods of the present invention using the methods described in Example 1. MC4R agonists which are useful in the present invention generally have an IC₅₀ less than 100 nM in the MC4R agonist binding assay and an EC₅₀ less than 100 nM in the functional assay described in Example 2. Particularly useful in the present invention are MC4R agonists with an IC₅₀ less than 45 nM or an EC₅₀ less than 15 nM. More particularly useful in the present in invention are MC4R agonists with an EC₅₀ less than 15 mM and an IC₅₀ less than 45 nM.

Throughout the instant application, the following terms have the indicated meanings:

The alkyl groups specified above are intended to include those alkyl groups of the designated length in either a straight or branched configuration. Exemplary of such alkyl groups are methyl, ethyl, propyl, isopropyl, butyl, sec-butyl, tertiary butyl, pentyl, isopentyl, hexyl, isohexyl, and the like.

The term “halogen” is intended to include the halogen atoms fluorine, chlorine, bromine and iodine.

The term “C₁₋₄ alkyliminoyl” means C₁₃C(═NH)—.

The term “aryl” includes phenyl and naphthyl.

The term “heteroaryl” includes mono- and bicyclic aromatic rings containing from 1 to 4 heteroatoms selected from nitrogen, oxygen and sulfur. “5- or 6-Membered heteroaryl” represents a monocyclic heteroaromatic ring; examples thereof include thiazole, oxazole, thiophene, furan, pyrrole, imidazole, isoxazole, pyrazole, triazole, thiadiazole, tetrazole, oxadiazole, pyridine, pyridazine, pyrimidine, pyrazine, and the like. Bicyclic heteroaromatic rings include, but are not limited to, benzothiadiazole, indole, benzothiophene, benzofuran, benzimidazole, benzisoxazole, benzothiazole, quinoline, benzotriazole, benzoxazole, isoquinoline, purine, furopyridine and thienopyridine.

The term “5- or 6-membered carbocyclyl” is intended to include non-aromatic rings containing only carbon atoms such as cyclopentyl and cyclohexyl.

The term “5 and 6-membered heterocyclyl” is intended to include non-aromatic heterocycles containing one to four heteroatoms selected from nitrogen, oxygen and sulfur. Examples of a 5 or 6-membered heterocyclyl include piperidine, morpholine, thiamorpholine, pyrrolidine, imidazolidine, tetrahydrofuran, piperazine, and the like.

Certain of the above defined terms may occur more than once in the above formula and upon such occurrence each term shall be defined independently of the other; thus for example, NR⁴R⁴ may represent NH₂, NHCH₃, N(CH₃)CH₂CH₃, and the like.

It will be understood that, as used herein, references to MC4R agonists, including MC4R agonists of Formulas I and II, and compound A, are meant to also include the pharmaceutically acceptable salts and esters thereof.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, lithium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, formic, fumaric, gluconic, glutamic, hydrobromic, hydrochloric, isethionic, lactic, maleic, malic, mandelic, methanesulfonic, malonic, mucic, nitric, pamoic, pantothenic, phosphoric, propionic, succinic, sulfuric, tartaric, p-toluenesulfonic acid, trifluoroacetic acid, and the like. Particularly preferred are citric, fumaric, hydrobromic, hydrochloric, maleic, phosphoric, sulfuric, and tartaric acids. It will be understood that, as used herein, references to the compounds of Formula I, formula II, compound A, and other melanocortin 4 receptor agonists, are meant to also include the pharmaceutically acceptable salts, such as the hydrochloride salts.

The compounds useful in the methods of the present invention include stereoisomers, such as optical isomers, diastereomers and geometerical isomers, or tautomers depending on the mode of substitution. The compounds may contain one or more chiral centers and occur as racemates, racemic mixtures and as individual diastereomers, enantiomeric mixtures or single enantiomers, keto-enol tautomers, or E and Z olefinic geometric isomers, with all isomeric forms being included in the present invention. The present invention is meant to comprehend all such isomeric forms of the compounds in the compositions of the present invention, and their mixtures. Therefore, where a compound is chiral, the separate enantiomers, substantially free of the other, are included within the scope of the invention; further included are all mixtures of the two enantiomers. Also included within the scope of the invention are polymorphs, hydrates and solvates of the compounds of the instant invention.

The present invention includes within its scope prodrugs of the compounds in the compositions of this invention. In general, such prodrugs will be functional derivatives of the compounds in these compositions which are readily convertible in vivo into the required compound. Thus, in the methods of treatment of the present invention, the term “administering” shall encompass the treatment of substance addiction, substance abuse, substance addiction-relate disorders, nicotine addiction, nicotine abuse, and nicotine addiction-related disorders with the compounds specifically disclosed as elements of the composition or with compounds which may not be specifically disclosed, but which convert to the specified compounds in vivo after administration to the patient. Conventional procedures for the selection and preparation of suitable prodrug derivatives are described, for example, in “Design of Prodrugs,” ed. H. Bundgaard, Elsevier, 1985.

Utility

The compounds of the present invention are useful to inhibit or reduce voluntary substance consumption, and for the treatment or prevention of substance abuse, substance addiction, and substance addiction-related disorders. In particular, the compounds of the present invention are useful to inhibit or reduce voluntary nicotine consumption, and for the treatment or prevention of nicotine abuse, nicotine addiction and nicotine addiction related disorders.

The term “substance” as referred to herein is understood to mean a drug such as nicotine, marijuana, cocaine, an amphetamine and an opiate. In one embodiment, the term substance refers to nicotine.

Substance addiction is a chronic, relapsing disease characterized by compulsive, and at times uncontrollable physical or psychological craving for a particular substance, substance seeking and substance use, that persists despite negative consequences, and accompanied by physical dependence, wherein the body is adapted to the existing substance, and/or psychological dependence on the substance. Addiction is characterized by repeated self-administration that typically results in tolerance, withdrawal, compulsive drug taking behavior, drug seeking behavior, and relapse. The compulsion to seek and use drugs can take over the individual's life, resulting in dysfunctional behaviors that can interfere with normal functioning in the family, workplace and broader community. Addiction places people at risk for a variety of other illnesses that result from poor living conditions and health habits, as well as the toxic effects of drugs themselves.

Substance addiction includes, but is not limited to, an addiction to drugs such as nicotine, cocaine, marijuana, amphetamines and opiates. Substances which may result primarily in psychological dependence or addiction include amphetamines, marijuana, cocaine, and opiates.

Substances which result primarily in physical dependence or addiction are nicotine, and opiates.

Substance addiction-related disorders include, but are not limited to, disorders resulting from substance use, substance abuse, and substance addiction, such as cocaine addiction and abuse related disorders; marijuana abuse and addiction related disorders, amphetamine abuse and addiction related disorders, and opiate addiction and abuse related disorders. For example, substance addition-related disorders include drug withdrawal disorders such as amphetamine withdrawal; cocaine withdrawal; opioid withdrawal; marijuana withdrawal; and withdrawal symptoms due to other substances; and substance withdrawal induced anxiety, mood and sleep disorders.

Substance abuse is the excessive use of a substance or use for purposes other than those for which they are indicated or in a manner or quantities other than directed. Harmful substance use implies substance use that causes health consequences, such as physical or mental damage.

The term “nicotine” as used herein refers to nicotine contained in tobacco and other naturally occurring sources, as well as synthetic nicotine, and salts thereof, including but not limited to, the salicylate or bitartrate salt thereof.

Nicotine abuse is characterized by excessive use of nicotine, even in the face of negative health consequences.

Nicotine addiction is a destructive pattern of nicotine use, leading to significant social occupational, or medical impairment and characterized by three or more of the following symptoms: 1) nicotine tolerance (a need for markedly increased amounts of nicotine to achieve intoxication, or markedly diminished effect with continued use of the same amount of nicotine); 2) nicotine withdrawal symptoms (sweating or rapid pulse, increased hand tremor, insomnia, nausea or vomiting, physical agitation, anxiety, transient visual, tactile, or auditory hallucinations or illusions, grand mal seizures), 3) nicotine administration to relieve or avoid withdrawal symptoms, 4) greater use than nicotine than intended, 5) unsuccessful efforts to cut down or control nicotine use, 6) persistent desire or unsuccessful efforts to cut down or control nicotine use, 7) great deal of time spent using nicotine, 8) nicotine caused reduction in social, occupational or recreational activities, and 9) continued use of nicotine despite knowledge of having a persistent or recurrent physical or psychological problem that is likely to have been worsened by nicotine use.

Nicotine addiction-related disorders include, but are not limited to: cancer of the lung, mouth, pharynx, larynx, esophagus, cervix, kidney, ureter and bladder; chronic bronchitis; emphysema; asthma; heart disease, including stroke, heart attack, vascular disease, and aneurysm; premature delivery; spontaneous abortion; and infants with decreased birth weight; as well as nicotine withdrawal symptoms.

Cocaine addiction and abuse related disorders include, but are not limited to: hyperstimulation, tachycardia, hypertension, mydriasis, muscle twitching, sleeplessness, extreme nervousness, hallucinations, paranoid delusions, aggressive behavior, increased heart and respiration rates, increase blood pressure and depression when use is discontinued. Cocaine overdose may produce tremors, convulsions, delirium and death resulting from heart arrhythmias and cardiovascular failure.

Marijuana abuse and addiction related disorders include, but are not limited to: tachycardia, conjunctival injection, dry mouth, panic, decrease in communicative and motor abilities, depth perception and tracking impairment, altered sense of timing, exacerbated schizophrenic symptoms, increase in appetite, acute bronchitis, wheezing, coughing, increased phlegm, and altered pulmonary function.

Amphetamine abuse and addiction related disorders include, but are not limited to: EEG changes, fatigue, and mental depression on withdrawal, tachycardia, auditory and visual hallucinations, delusions, anxiety reactions, paranoid psychosis, exhaustion syndrome, confusion, memory loss, prolonged depression with suicidal tendencies.

Opiate addiction and abuse related disorders, such as those resulting from heroin and methadone use, include, but are not limited to: withdrawal syndrome (CNS hyperactivity, anxiety, craving, increased respiratory rate, perspiration, lacrimation, rhinorrhea, mydriasis, piloerection, tremors, muscle twitching, hot and cold flashes, aching muscles and anorexia), cutaneous abscesses, cellulitis, lymphangitis, lymphadenitis, phlebitis, deep muscle aches, and overdose. Complications from heroin addiction include pulmonary disorders (aspiration pneumonitits, pneumonia, lung abcess, septic pulmonary emboli and atelectasis, hepatitis, arthritic disorders, osteomyelitis in lumbar vertebral region, infectious spondylitis, sacroiliitis, myositis ossificans, and extraosseous metaplasia), immunologic (hypergammaglobulinemia of IgG and IgM, HIV infection, and AIDS) and neurologic disorders (toxic amblyopia, transverse myelitis, mononeuropathies, polyneuropathies, Guillian-Barre syndrome, bacterial meningitis, mycotic aneurysm, brain abscess, subdural and epidural abscesses).

The term “opiate(s)” as used herein includes, but is not limited to, heroin; narcotics, such as morphine; opium; codeine; oxycodone (Oxycontin®); propoxyphene (Darvon®); hydrocodone (Vicodin®), hydromorphone (Dilaudid®); meperidine (Demerol®), and Lomotil®.

The term “amphetamine(s)” as used herein includes, but is not limited to, amphetamine, dextroamphetamine, and methamphetamine.

Relapse is a return to substance use, or nicotine use, after a period of abstinence. Factors that “precipitate” relapse after a period of abstinence include, stress, exposure to the drug of abuse, and exposure to a cue or a context associated with the administration of the addictive agent.

The term “physical dependence” as used herein means an adaptive state produced by repeated substance administration and is accompanied by withdrawal symptoms, wherein physiological changes occur when the substance is discontinued.

The term psychological dependence” as used herein means a condition characterized by an intense urge, drive or craving to use the drug whose effects the user feels are necessary for a sense of well being. Psychological dependence is accompanied by feelings of satisfaction and a desire to repeat the drug experience or to avoid the discontent of not having it.

The term “tolerance” as used herein means the need to increase the dose of a drug after several administrations to achieve the same magnitude of effect.

“Treatment” (of nicotine abuse and nicotine addiction) refers to the administration of the compounds or combinations of the present invention to reduce or inhibit the use of nicotine by a subject. One outcome of treatment may be reducing the use of nicotine in a subject relative to the subject's nicotine use prior to treatment. Another outcome of treatment may be inhibiting the use of nicotine in a subject. Another outcome of treatment may be decreasing the occurrence of nicotine intake in a subject. Another outcome of treatment may be decreasing the severity of nicotine intake, such as decreasing the amount of nicotine consumed, in a subject. Another outcome of treatment may be to administer the compounds or combinations of the present invention to reduce or inhibit the consumption of nicotine in a subject in need thereof. Another outcome of treatment is to reduce or inhibit nicotine withdrawal symptoms. Another outcome of treatment is to reduce or inhibit nicotine craving. Another outcome of treatment is to block the reinforcing effect of nicotine.

The term “inhibit” nicotine consumption means to decrease nicotine consumption in a subject. One outcome of inhibition may be to stop nicotine consumption in a subject in need thereof.

The term “reduce nicotine consumption” or “reduction of nicotine consumption” means to decrease the amount of nicotine consumed by a subject relative to the amount of nicotine consumed prior to the start of treatment. In one embodiment the amount of nicotine consumed by a subject is decreased by at least 10% relative to the amount of nicotine consumed prior to the start of treatment. In another embodiment, the amount of nicotine consumed by a subject is decreased by at least 25% relative to the amount of nicotine consumed prior to the start of treatment. In another embodiment, the amount of nicotine consumed by a subject is decreased by at least 67% relative to the amount of nicotine consumed prior to the start of treatment. In yet another embodiment, the amount of nicotine consumed by a subject is decreased by at least 80% relative to the amount of nicotine consumed prior to the start of treatment.

“Prevention” (of nicotine abuse and nicotine addiction) refers to the administration of the compounds or combinations of the present invention to prevent nicotine abuse, nicotine addiction or developing a nicotine addiction-related disorder in a subject by administration prior to the start of nicotine use. One outcome of prevention may be to prevent nicotine use in a subject by administration prior to the start of nicotine use. Another outcome of prevention may be to prevent nicotine abuse in a subject. Another outcome of prevention may be to prevent nicotine addiction in a subject. Another outcome of prevention may be to prevent the development of a nicotine addiction related disorder in a subject. Another outcome of prevention may be preventing nicotine use from occurring if the treatment is administered prior to the onset of nicotine use in a subject. Another outcome of prevention may be to prolong resistance to nicotine use in a subject. Another outcome of prevention may be to administer the compounds or combinations of the present invention to prevent nicotine use in a subject at risk of nicotine abuse, nicotine addiction or developing a nicotine addiction-related disorder in a subject. Another outcome of prevention is to prevent nicotine withdrawal symptoms when drug use is stopped. Another outcome of prevention is to prevent nicotine craving when nicotine use is stopped. Another outcome of prevention is to block the reinforcing effect of nicotine before nicotine use is stopped.

Moreover, if treatment is commenced in a subject already consuming nicotine, such treatment may prevent the occurrence, progression or severity of nicotine addiction-related disorders, such as, but not limited to, cancer of the lung, mouth, pharynx, larynx, esophagus, cervix, kidney, ureter and bladder; chronic bronchitis; emphysema; asthma; heart disease, including stroke, heart attack, vascular disease, and aneurysm; premature delivery; spontaneous abortion; and infants with decreased birth weight; as well as nicotine withdrawal symptoms.

“Treatment” (of substance abuse or addiction) refers to the administration of the compounds or combinations of the present invention to reduce or inhibit the use of the substance by a subject. One outcome of treatment may be reducing the use of the substance in a subject relative to the subject's substance use prior to treatment. Another outcome of treatment may be inhibiting the use of the substance in a subject. Another outcome of treatment may be decreasing the occurrence of substance intake in a subject. Another outcome of treatment may be decreasing the severity of substance intake, such as decreasing the amount of the substance consumed, in a subject. Another outcome of treatment may be to administer the compounds or combinations of the present invention to reduce or inhibit the consumption of the substance in a subject in need thereof. Another outcome of treatment is to reduce or inhibit substance withdrawal symptoms. Another outcome of treatment is to reduce or inhibit substance craving. Another outcome of treatment is to block the reinforcing effect of the substance.

The term “inhibit” substance consumption means to reduce substance consumption in a subject. One outcome of inhibition may be to stop substance consumption in a subject in need thereof.

The term “reduce substance consumption” or “reduction of substance consumption” means to decrease the amount of the substance consumed by a subject relative to the amount of the substance consumed prior to the start of treatment. In one embodiment the amount of substance consumed by a subject is decreased by at least 10% relative to the amount of substance consumed prior to the start of treatment. In another embodiment, the amount of substance consumed by a subject is decreased by at least 25% relative to the amount of substance consumed prior to the start of treatment. In another embodiment, the amount of substance consumed by a subject is decreased by at least 67% relative to the amount of substance consumed prior to the start of treatment. In yet another embodiment, the amount of substance consumed by a subject is decreased by at least 80% relative to the amount of substance consumed prior to the start of treatment.

“Prevention” (of substance abuse and substance addiction) refers to the administration of the compounds or combinations of the present invention to prevent substance abuse, substance addiction or developing a substance addiction-related disorder in a subject by administration prior to the start of substance use. One outcome of prevention may be to prevent substance use in a subject by administration prior to the start of substance use. Another outcome of prevention may be to prevent substance abuse in a subject. Another outcome of prevention may be to prevent substance addiction in a subject. Another outcome of prevention may be to prevent the development of a substance addiction related disorder in a subject. Another outcome of prevention may be preventing substance use from occurring if the treatment is administered prior to the onset of substance use in a subject. Another outcome of prevention may be to prolong resistance to substance use in a subject. Another outcome of prevention may be to administer the compounds or combinations of the present invention to prevent substance use in a subject at risk of substance abuse, substance addiction or developing a substance addiction-related disorder in a subject. Another outcome of prevention is to prevent substance withdrawal symptoms when substance use is stopped. Another outcome of prevention is to prevent substance craving when substance use is stopped. Another outcome of prevention is to block the reinforcing effect of the substance before substance use is stopped.

Moreover, if treatment is commenced in a subject already consuming the substance, such treatment may prevent the occurrence, progression or severity of substance addiction-related disorders.

The term “selective” means having an activation preference for a specific receptor over other receptors which can be quantified based upon whole cell, tissue, or organism assays which demonstrate receptor activity, such as the cAMP Functional Assay and the Binding Affinity Assay. The compounds of the present invention interact preferentially (i.e. selectively) with the MC-4 receptor relative to the other melanocortin receptors. Selectivity for the MC-4 receptor is beneficial for compounds administered to humans or mammals, to minimize the number of side effects associated with their administration. MC-4 selectivity of a compound over another MC receptor is defined herein as the EC₅₀, or IC₅₀, of the compound at the MC receptor being referenced over the EC₅₀, or IC₅₀, of the compound for the MC-4 receptor. As used herein, unless indicated otherwise, use of the term “selective over the other MC receptors” means selective with respect to the other melanocortin receptors, including the MC-1, MC-2, MC-3 and MC-5 receptors. For example, a compound having an EC₅₀ of 8 nM at the MC-4 receptor and an EC₅₀ of >80 nM at the MC-1, MC-2 MC-3, and MC-5 receptors has a selectivity ratio for the MC-4 receptor over the other MC receptors of at least 1:10. Additionally, the term “selective” may also refer to one of the MC-1, MC-2, MC-3 or MC-5 receptors individually. For example, a compound having an EC₅₀ of 8 nM at the MC-4 receptor and an EC₅₀ of 80 nM at the MC-1 receptor has a selectivity ratio for the MC-4 receptor over the MC-1 receptor of 1:10. Such a compound is selective over the MC-1 receptor regardless of its EC₅₀ value for the MC-2R or MC-5R. For example, the selectivity of a compound for the MC-4R over the MC-1R is defined as: MC-4R functional selectivity, EC₅₀=[EC₅₀ MC-1R]/[EC₅₀ MC-4R], or MC-4R binding selectivity, IC₅₀=[IC₅₀ MC-1R]/[IC₅₀ MC-4R]. A compound is defined herein as being “selective” for the MC-4 receptor when the above mentioned ratio is at least 10, preferably at least 65, and more preferably at least about 100.

The terms “administration of” and or “administering” a compound should be understood to mean providing a compound of the invention or a prodrug of a compound of the invention to a subject. The instant pharmaceutical composition includes administration of a single pharmaceutical dosage formulation which contains the melanocortin 4 receptor agonist.

The term “subject”, as used herein refers to an animal, preferably a mammal, more preferably a human. In one embodiment of the term “mammal in need thereof” is a “human in need thereof,” said human being either male or female. In another embodiment of the present invention, the term “subject” refers to a human that is or has been the object of treatment, observation or experiment. In another embodiment of the present invention, the term “subject” refers to a “subject in need thereof”. In a class of this embodiment, the “subject in need thereof” refers to a subject who is in need of treatment or prophylaxis as determined by a researcher, veterinarian, medical doctor or other clinician. In another embodiment, the “subject in need thereof” is a human that is addicted to a substance. In a class of this embodiment, the human is addicted to nicotine. In another embodiment, the “subject in need thereof” is a human that is a substance abuser. In a class of this embodiment, the human abuses nicotine. In another embodiment, the “subject in need thereof” is a human that uses a substance. In a class of this embodiment, the human uses nicotine. In another embodiment, the “subject in need thereof” has a substance addiction-related disorder. In a class of this embodiment, the human has a nicotine addiction related disorder. In another embodiment, the “subject in need thereof” is a subject in need of terminating a substance addiction. In another embodiment, the “subject in need thereof” is a subject in need of terminating a nicotine addiction. In another embodiment, the “subject in need thereof” is a subject who uses a substance on a regular basis. In a class of this embodiment, the subject is either unwilling or unable to terminate substance use. In another embodiment, the “subject in need thereof” is a subject who uses nicotine on a regular basis. In a class of this embodiment, the subject is either unwilling or unable to terminate nicotine use.

In yet another embodiment of the present invention, the term “subject” refers to a “subject at risk thereof”. In a class of this embodiment, the “subject at risk thereof” is a subject at risk of developing a substance addiction. In a subclass of this class, the subject is at risk of developing nicotine addiction. In a class of this embodiment, the “subject at risk thereof” is a subject at risk of abusing a substance. In a subclass of this class, the subject is at risk of abusing nicotine. In a class of this embodiment, the “subject at risk thereof” is a subject at risk of using a substance. In a subclass of this class, the subject is at risk of using nicotine. In another class of this embodiment, the “subject at risk thereof” is a subject at risk of developing a substance addiction related disorder. In a subclass of this class, the subject is at risk of developing a nicotine addiction related disorder.

The administration of the composition of the present invention in order to practice the present methods of therapy is carried out by administering a therapeutically effective amount of the compounds in the composition to a subject in need of such treatment or prophylaxis. The need for a prophylactic administration according to the methods of the present invention is determined via the use of well known risk factors. The effective amount of an individual compound is determined, in the final analysis, by the physician in charge of the case, but depends on factors such as the exact disease to be treated, the severity of the disease and other diseases or conditions from which the patient suffers, the chosen route of administration, other drugs and treatments which the patient may concomitantly require, and other factors in the physician's judgment.

The term “therapeutically effective amount” as used herein means the amount of the active compounds in the composition that will elicit the biological or medical response in a tissue, system, subject, or human that is being sought by the researcher, veterinarian, medical doctor or other clinician, which includes alleviation of the symptoms of the disorder being treated. Disorders being treated include, but are not limited to, substance addiction, substance abuse, substance addiction-related disorders, nicotine addiction, nicotine abuse, and nicotine addiction-related disorders in subjects in need thereof. The novel methods of treatment of this invention are for disorders known to those skilled in the art.

The term “prophylactically effective amount” as used herein means the amount of the active compounds that will elicit the biological or medical response in a tissue, system, subject, or human that is being sought by the researcher, veterinarian, medical doctor or other clinician, to prevent the onset of substance addiction, substance abuse, substance addiction-related disorders, nicotine addiction, nicotine abuse, and nicotine addiction-related disorders in subjects as risk for substance addiction, substance abuse, substance addiction-related disorders, nicotine addiction, nicotine abuse, and nicotine addiction-related disorders.

The term “composition”, as in pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier.

By a melanocortin receptor “agonist” is meant an endogenous or drug substance or compound that can interact with a melanocortin receptor and initiate a pharmacological response characteristic of the melanocortin receptor. By a melanocortin receptor “antagonist” is meant a drug or a compound that opposes the melanocortin receptor-associated responses normally induced by another bioactive agent. The “agonistic” properties of the compounds of the present invention were measured in the functional assay described below. The functional assay discriminates a melanocortin receptor agonist from a melanocortin receptor antagonist.

By “binding affinity” and “binding affinity index” is meant the ability of a compound/drug to bind to its biological target, in the present instance, the ability of a melanocortin 4 receptor agonist, including compounds of structural formula I and II, to bind to a melanocortin receptor. Binding affinities for the compounds of the present invention were measured in the binding assay described below and are expressed as IC₅₀ values.

“Efficacy” describes the relative intensity with which agonists vary in the response they produce even when they occupy the same number of receptors and with the same affinity. Efficacy is the property that enables drugs to produce responses. Properties of compounds/drugs can be categorized into two groups, those which cause them to associate with the receptors (binding affinity) and those that produce a stimulus (efficacy). The term “efficacy” is used to characterize the level of maximal responses induced by agonists. Not all agonists of a receptor are capable of inducing identical levels of maximal responses. Maximal response depends on the efficiency of receptor coupling, that is, from the cascade of events, which, from the binding of the drug to the receptor, leads to the desired biological effect.

The functional activities expressed as EC₅₀'s and the “agonist efficacy” for the compounds of the present invention at a particular concentration were measured in the functional assay described below.

The magnitude of prophylactic or therapeutic dose of the active ingredient (the MC4R agonist) will, of course, vary with the nature of the severity of the condition to be treated and with the particular compound and its route of administration. It will also vary according to the age, weight and response of the individual patient. In general, the daily dose range of each compound lies within the range of from about 0.001 mg/kg to about 1000 mg/kg; more specifically from about 0.001 mg/kg to about 100 mg/kg body weight of a subject per day in single or divided doses. On the other hand, it may be necessary to use dosages outside these limits in some cases.

For use where a composition for intravenous administration is employed, a suitable dosage range is from about 0.001 mg/kg to about 1000 mg/kg; more specifically from 0.001 mg/kg to about 100 mg/kg of each compound in the composition per day.

In the case where an oral composition is employed, a suitable dosage range is, e.g. from about 0.001 mg/kg to about 1000 mg/kg of each compound in the composition per day; more specifically from about 0.001 mg/kg to about 100 mg/kg per day. For oral administration, the compositions are provided in the form of tablets containing from 0.01 mg to 1,000 mg; more specifically 0.01, 0.05, 0.1, 0.2, 0.5, 1.0, 2.5, 5, 10, 15, 20, 25, 30, 40, 50, 75, 100, 125, 150, 175, 200, 225, 250, 500, 650, 750, 850 and 1,000 milligrams of each active ingredient for the symptomatic adjustment of the dosage to the subject to be treated. This dosage regimen may be adjusted to provide the optimal therapeutic response.

The compounds of this invention can be administered to humans in the dosage ranges specific for each compound. In general, for treating, preventing, reducing or inhibiting substance addiction, substance abuse, and substance addiction-related disorders, including blocking the reinforcing effect of the substance, the MC4R agonist is administered at a daily dosage of from about 0.001 mg/kg to about 1000 mg/kg of body weight orally. More specifically, when treating, preventing, reducing or inhibiting nicotine addiction, nicotine abuse and nicotine addiction-related disorders, including blocking the reinforcing effect of nicotine, generally satisfactory results may be obtained when a MC4R agonist, such as a MC4R agonist of Formula I, Formula II and Compound A, or a pharmaceutically acceptable salt or ester thereof, is administered at a daily oral dosage of from about 0.001 mg/kg to about 1000 mg/kg; more specifically from about 0.001 mg/kg to about 100 mg/kg of body weight, given in a single dose or in divided doses two to six times a day, or in sustained release form. In the case of a 70 kg adult human, the total daily dose will generally be from about 0.07 milligrams to about 3500 milligrams.

The effective dosage of the active ingredient employed in the composition may vary depending on the particular compound employed, the mode of administration, the condition being treated and the severity of the condition being treated. Thus, the dosage regimen utilizing the compositions of the present invention is selected in accordance with a variety of factors including type, species, age, general health, body weight, diet, sex and medical condition of the subject; the severity of the condition to be treated; the renal and hepatic function of the patient; the drug combination; and the particular compound employed and its route of administration. A physician, clinician or veterinarian of ordinary skill can readily determine and prescribe the effective amount of the drug required to prevent, counter or arrest the progress of the condition.

Combination Therapy

The melanocortin 4 receptor agonists useful in the methods of the present invention may be used in combination with other drugs that are used in the treatment or prevention of substance addiction, substance abuse and substance abuse-related disorders, in particular nicotine addiction, nicotine abuse and nicotine abuse related disorders. Such other drugs may be administered, by a route and in an amount commonly used therefor, contemporaneously or sequentially with a melanocortin 4 receptor agonist. When a melanocortin 4 receptor agonist is used contemporaneously with one or more other drugs, a pharmaceutical composition containing such other drugs in addition to the melanocortin 4 receptor agonist is preferred. Accordingly, the pharmaceutical compositions useful in the methods of the present invention include those that also contain one or more other active ingredients, in addition to a melanocortin 4 receptor agonist.

Examples of other active ingredients that may be combined with the melanocortin 4 receptor agonists useful in the present invention for smoking cessation, and for the treatment or prevention of substance addiction or dependence, substance abuse and substance abuse-related disorders, either administered separately or in the same pharmaceutical compositions, include, but are not limited to:

(a) cannabinoid CB-1 antagonists/inverse agonists, such as rimonabant;

(b) antidepressants, such as bupropion, doxepine, or nortriptyline;

(c) opioid antagonists, such as naloxone, naltrexone, and nalmefene;

(d) inhibitors of acetaldehye metabolism, such as disulfuram;

(e) CNS stimulants, such as methylphenidate;

(f) dopamine D3 antagonists/inverse agonists; and

(g) alpha adrenergic agents;

(h) methadone;

or another active ingredient demonstrating efficacy in aiding cessation of substance consumption.

Examples of other active ingredients that may be combined with the melanocortin 4 receptor agonists useful in the present invention for smoking cessation, and for the treatment or prevention of nicotine addiction or dependence, nicotine abuse and nicotine abuse related disorders; for the prevention of craving and relapse, either administered separately or in the same pharmaceutical compositions, include, but are not limited to:

(a) cannabinoid CB-1 antagonists/inverse agonists, such as rimonabant;

(b) nicotine receptor agonists or nicotine receptor partial agonists;

(c) anti-depressants, such as bupropion, doxepine, or nortriptyline;

(d) dopamine D3 antagonists/inverse agonists, such as BP-897 (Bioproject), SB-277011-A, SB-269652, Sb-414796 (Glaxo Smithkline), AVE-5997 (Aventis) PNU-177864 (Pfizer), S-14297, S-33084, S-33138 (Servier);

(e) monoamine oxidase inhibitors;

(f) anxiolytics, such as buspirone or clonidine;

(g) glutamate antagonists, such as LY-354740;

(h) glycine antagonists, such as GW 468816;

(i) a nicotine vaccine, such as TA-NIC, and NicVax,

(j) N-methyl-D-aspartate receptor subunit 2B antagonists, such as GW-468816;

(k) GABA-B agonists/potentiators, such as Baclofen;

(l) muscarinic M5 receptor antagonists,

(m) mGluR5 Inverse agonists;

(n) Zyban XL; and

(O) varenicline;

or another active ingredient demonstrating efficacy in aiding cessation of tobacco consumption.

For the treatment and prevention of nicotine addiction, nicotine abuse, and nicotine addiction related disorders; to prevent nicotine withdrawal symptoms, to reduce nicotine craving, to block the reinforcing effect of nicotine, and to facilitate smoking cessation: the melanocortin 4 receptor agonist may be combined with other nicotine therapies, including but not limited to, nicotine replacement therapies, such as Nicoderm®, Nicoderm®, Nicorette®, nicotine nasal spray, nicotine inhaler, as well as with nicotine replacement therapies, such as Zyban, Doxepine, Imipramine, and Desipramine.

For the treatment of opiate abuse, opiate addiction, and to alleviate opiate withdrawal symptoms, the melanocortin 4 receptor agonist may be combined with: 1) an opiate replacement therapy, such as methadone, 2) an opiate antagonist such as Naltrexone, Medisorb Naltrexone, Depotrex (Biotek) and Buprenorphine Depot (Drug Abuse Sciences); 3) an alpha 2 adrenergic agonist, such as Lofexedine (Forum); and 4) a hydromorphone inhibitor, such as Naltrex (Drug Abuse Sciences); and 5) a non-narcotic analgesic, such as Tetrodin (Int. Wex).

For the treatment of cocaine abuse, cocaine addiction, to alleviate cocaine withdrawal symptoms, to reduce craving, and to prevent cocaine addiction related disorders, the melanocortin 4 receptor agonist may be combined with: 1) a non-narcotic analgesic, such as Tetrodin (Int. Wex), 2) a mixed monoamine reuptake inhibitor such as NS-2359 (Neurosearch); 3) a polyclonal antibody, such as COC-Ab (Drug Abuse Sciences); and 4) a vaccine, such as TA-CD (Xenova), or GNC-KLH (Scripps).

Additionally, the use of combination therapy in the methods of the present invention, comprising administering a therapeutically effective amount of a melanocortin 4 receptor agonist with a therapeutically effective or subtherapeutically effective amount, of a medication approved to treat or prevent substance abuse and addiction, in particular a medication approved to treat or prevent nicotine abuse and addiction, that acts via a different complementary mechanism, will result in improved efficacy.

It will be understood that the scope of compositions of the compounds of this invention with other agents useful to inhibit or reduce substance or nicotine consumption, and for treating or preventing substance addiction, substance abuse, substance addiction related disorders, nicotine addiction, nicotine abuse, or nicotine addiction related disorders, includes in principle any combination with any pharmaceutical composition useful to treat or prevent substance addiction, nicotine addiction, substance addiction-related disorders and nicotine addiction-related disorders.

Pharmaceutical Compositions

Another aspect of the present invention provides pharmaceutical compositions useful for the methods of the present invention comprising a pharmaceutical carrier and a therapeutically effective amount of a melanocortin-4 receptor agonist and each additional compound in the composition of the present invention. The term “composition”, as in pharmaceutical composition, is intended to encompass a product comprising the active ingredient(s), and the inert ingredient(s), such as pharmaceutically acceptable excipients, that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a melanocortin 4 receptor agonist, additional active ingredient(s), and/or pharmaceutically acceptable excipients and carriers.

Any suitable route of administration may be employed for providing a subject, especially a human, with an effective dosage of a composition of the present invention. For example, oral delivery, rectal delivery, topical delivery, parenteral delivery, ocular delivery, pulmonary delivery, nasal delivery, delivery to the central nervous system, in particular the brain, and the like may be employed. Dosage forms include tablets, troches, dispersions, suspensions, solutions, capsules, creams, ointments, aerosols, a mouth wash, a nasal spray, a toothpaste, a toothpick, a chewing gum, a solid dose that effervesces in the mouth, a candy, such as a chocolate or caramel chew, a composition licked from a stamp or other support material, a beverage additive, and the like.

The pharmaceutical compositions of the present invention comprise a MC4R agonist, as active ingredient or a pharmaceutically acceptable salt or ester thereof, and may also contain a pharmaceutically acceptable carrier and optionally other therapeutic ingredients. By “pharmaceutically acceptable” it is meant the carrier, diluent or excipient must be compatible with the other ingredients of the formulation and not deleterious to the recipient thereof. In particular, the term “pharmaceutically acceptable salts” refers to salts prepared from pharmaceutically acceptable non-toxic bases or acids including inorganic bases or acids and organic bases or acids.

The compositions include compounds suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. These compositions may be conveniently presented in unit dosage form and prepared by any of the methods well-known in the art of pharmacy.

For administration by inhalation, the compositions of the present invention are conveniently delivered in the form of an aerosol spray or nasal spray presentation from pressurized packs or nebulizers or from pump spray containers. The compositions may also be delivered as powders which may be formulated and the powder composition may be inhaled with the aid of an insufflation powder inhaler device. The preferred delivery systems for inhalation are metered dose inhalation (MDI) aerosol, which may be formulated as a suspension or solution of the instant composition in suitable propellants, such as fluorocarbons or hydrocarbons and dry powder inhalation (DPI) aerosol, which may be formulated as a dry powder of the composition with or without additional excipients. Suitable topical formulations of the compositions of the present invention include transdermal devices, aerosols, creams, solutions, ointments, gels, lotions, dusting powders, and the like. The topical pharmaceutical compositions containing the compositions of the present invention ordinarily include about 0.005% to 5% by weight of the active compounds in admixture with a pharmaceutically acceptable vehicle. Transdermal skin patches useful for administering the compositions of the present invention include those well known to those of ordinary skill in that art. To be administered in the form of a transdermal delivery system, the dosage administration will, of course be continuous rather than intermittent throughout the dosage regimen. The compositions of the present invention can also be administered in the form of liposome delivery systems, such as small unilamellar vesicles, large unilamellar vesicles and multilamellar vesicles. Liposomes can be formed from a variety of phospholipids, such as cholesterol, sterylamine or phosphatidylcholines. Compositions of the present invention may also be delivered by the use of monoclonal antibodies as individual carriers to which the compound molecules are coupled. The compounds in these compositions may also be coupled with soluble polymers as targetable drug carriers. Such polymers can include polyvinylpyrrolidone, pyran copolymer, polyhydroxypropyl-methacrylamide phenol, polyhydroxyethylasparamidepheon, or polyethyleneoxidepolylysine substituted with palmitoyl residues. Furthermore, the compositions of the present invention may be coupled to a class of biodegradable polymers useful in achieving controlled release of a drug, for example, polylactic acid, polyepsilon caprolactone, polyhydroxybutyric acid, polyorthoesters, polyacetals, polydihydropyrans, polycyanoacrylates and cross-linked or amphipathic block copolymers of hydrogels.

In practical use, each compound in the compositions of the present invention (e.g. MC4R agonist) can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules, pellet, powder and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

In addition to the common dosage forms set out above, the composition may also be administered by controlled release means and/or delivery devices such as those described in U.S. Pat. Nos. 3,845,770; 3,916,899; 3,536,809; 3,598,123; 3,630,200 and 4,008,719, which are hereby incorporated by reference in their entirety.

For example, for oral administration in the form of a tablet, capsule, pellet, or powder, the active ingredient can be combined with an oral, non-toxic, pharmaceutically acceptable inert carrier such as lactose, starch, sucrose, glucose, methyl cellulose, magnesium stearate, mannitol, sorbitol, croscarmellose sodium and the like; for oral administration in liquid form, e.g., elixirs, syrups, slurries, emulsions, suspensions, solutions, and effervescent compositions, the oral drug components can be combined with any oral, non-toxic, pharmaceutically acceptable inert carrier such as ethanol, glycerol, water, oils and the like. Moreover, when desired or necessary, suitable binders, lubricants, disintegrating agents, buffers, coatings, and coloring agents can also be incorporated. Suitable binders can include starch, gelatin, natural sugars such a glucose, anhydrous lactose, free-flow lactose, beta-lactose, and corn sweeteners, natural and synthetic gums, such as acacia, guar, tragacanth or sodium alginate, carboxymethyl cellulose, polyethylene glycol, waxes, and the like. Lubricants used in these dosage forms include sodium oleate, sodium stearate, magnesium stearate, sodium benzoate, sodium acetate, sodium chloride and the like. Various other materials may be present as coatings or to modify the physical form of the dosage unit. For instance, tablets may be coated with shellac, sugar or both. A syrup or elixir may contain, in addition to the active ingredient, sucrose as a sweetening agent, methyl and propylparabens as preservatives, a dye and a flavoring such as cherry or orange flavor. When a dosage unit form is a capsule, it may contain, in addition to materials of the above type, a liquid carrier such as a fatty oil. The active compounds can also be administered intranasally as, for example, liquid drops or spray.

Desirably, each tablet contains from 0.01 to 1,000 mg, particularly 0.01, 0.05, 0.1, 0.2, 0.5, 1.0, 2.5, 5, 10, 15, 20, 25, 30, 40, 50, 75, 100, 125, 150, 175, 200, 225, 250, 500, 600, 750, 850 and 1,000 milligrams of each active ingredient in the composition of the present invention (e.g. MC4R agonist) for the symptomatic adjustment of the dosage to the subject to be treated; and each cachet or capsule contains from about 0.01 to 1,000 mg, particularly 0.01, 0.05, 0.1, 0.2, 0.5, 1.0, 2.5, 5, 10, 15, 20, 25, 30, 40, 50, 75, 100, 125, 150, 175, 200, 225, 250, 500, 600, 750, 850 and 1,000 milligrams of each active in the composition of the present invention (e.g. MC4R agonist) for the symptomatic adjustment of the dosage to the subject to be treated.

Exemplifying the invention is a pharmaceutical composition comprising a MC4R agonist described above and a pharmaceutically acceptable carrier. Also exemplifying the invention is a pharmaceutical composition made by combining any of the MC4R agonists described above and a pharmaceutically acceptable carrier. An illustration of the invention is a process for making a pharmaceutical composition comprising combining any of the MC4R agonists described above and a pharmaceutically acceptable carrier.

The dose may be administered in a single daily dose or the total daily dosage may be administered in divided doses of two to six times daily. Furthermore, based on the properties of the individual compound selected for administration, the dose may be administered less frequently, e.g., weekly, twice weekly, monthly, etc. The unit dosage will, of course, be correspondingly larger for the less frequent administration.

When administered via intranasal routes, transdermal routes, by rectal or vaginal suppositories, or through a continual intravenous solution, the dosage administration will, of course, be continuous rather than intermittent throughout the dosage regimen.

The following are examples of representative pharmaceutical dosage forms for the compositions of the present invention:

| Tablet | mg/tablet | |
|---|---|---|
| Compound A | 25 | |
| Microcrystalline Cellulose | 50.5 | |
| Lactose | 111.5 | |
| Croscarmellose Sodium | 5.0 | |
| Hydroxypropylcellulose | 6.0 | |
| Sodium Dodecyl Sulfate | 1.0 | |
| Magnesium Stearate | 1.0 | |
| | 200 | |
| Capsule | mg/capsule | |
| Compound A | 100 | |
| Lactose | 80 | |
| Sodium Dodecyl Sulfate | 20 | |
| | 200 | |
| Aerosol | Per canister | |
| Compound A | 13 | mg |
| Lecithin, NF Liq. Conc. | 1.2 | mg |
| Trichlorofluoromethane, NF | 4.65 | g |
| Dichlorodifluoromethane, NF | 12.15 | g |
| | 30 | |

In order to illustrate the invention, the following examples are included. These examples do not limit the invention. They are only meant to suggest a method of reducing the invention to practice. Those skilled in the art may find other methods of practicing the invention which are readily apparent to them. However, those methods are also deemed to be within the scope of this invention.

### EXAMPLE 1

### Binding Assay

The membrane binding assay was used to identify competitive inhibitors of ¹²⁵I-NDP-alpha-MSH binding to cloned human MCRs expressed in mouse L- or Chinese hamster ovary (CHO)-cells.

Cell lines expressing melanocortin receptors were grown in T-180 flasks containing selective medium of the composition: 1 L Dulbecco's modified Eagles Medium (DMEM) with 4.5 g L-glucose, 25 mM Hepes, without sodium pyruvate, (Gibco/BRl); 100 ml 10% heat-inactivated fetal bovine serum (Sigma); 10 mL 10,000 unit/mL penicillin & 10,000 μg/mL streptomycin (Gibco/BRl); 10 ml 200 mM L-glutamine (Gibco/BRl); 1 mg/mL geneticin (G418) (Gibco/BRl). The cells were grown at 37° C. with CO₂ and humidity control until the desired cell density and cell number was obtained.

The medium was poured off and 10 mls/T-180 flask of enzyme-free dissociation media (Specialty Media Inc.) was added. The cells were incubated at 37° C. for 10 min or until cells sloughed off when flask was banged against hand.

The cells were harvested into 200 mL centrifuge tubes and spun at 1000 rpm, 4° C., for 10 min. The supernatant was discarded and the cells were resuspended in 5 mls/monolayer membrane preparation buffer having the composition: 10 mM Tris pH 7.2-7.4; 4 μg/mL Leupeptin (Sigma); 10 μM Phosphoramidon (Boehringer Mannheim); 40 μg/mL Bacitracin (Sigma); 5 μg/mL Aprotinin (Sigma); 10 mM Pefabloc (Boehringer Mannheim). The cells were homogenized with motor-driven dounce (Talboy setting 40), using 10 strokes and the homogenate centrifuged at 6,000 rpm, 4° C., for 15 min.

The pellets were resuspended in 0.2 mls/monolayer membrane prep buffer and aliquots were placed in tubes (500-1000 μL/tube) and quick frozen in liquid nitrogen and then stored at −80° C.

Test compounds or unlabelled NDP-(x-MSH was added to 100 μL of membrane binding buffer to a final concentration of 1 μM. The membrane binding buffer had the composition: 50 mM Tris pH 7.2; 2 mM CaCl₂; 1 mM MgCl₂; 5 mM KCl; 0.2% BSA; 4 μg/mL Leupeptin (SIGMA); 10 μM Phosphoramidon (Boehringer Mannheim); 40 μg/mL Bacitracin (SIGMA); 5 μg/mL Aprotinin (SIGMA); and 10 mM Pefabloc (Boehringer Mannheim). One hundred μL of membrane binding buffer containing 10-40 μg membrane protein was added, followed by 100 μM [25]-NDP-α-MSH to final concentration of 100 μM. The resulting mixture was vortexed briefly and incubated for 90-120 min at room temp while shaking.

The mixture was filtered with Packard Microplate 196 filter apparatus using Packard Unifilter 96-well GF/C filter with 0.1% polyethyleneimine (Sigma). The filter was washed (5 times with a total of 10 mL per well) with room temperature of filter wash having the composition: 50 mM Tris-HCl pH 7.2 and 20 mM NaCl. The filter was dried, and the bottom sealed and 50 μL of Packard Microscint-20 was added to each well. The top was sealed and the radioactivity quantitated in a Packard Topcount Microplate Scintillation counter.

Melanocortin-4 receptor agonists of use in the present invention are compounds which are potent melanocortin-4 receptor agonists, i.e. compounds with an MC4R affinity (IC₅₀) of less than 300 nM, preferably less than 100 nM, and more preferably less than 45 nM.

Results of binding assay (Example 1) and selectivity for representative compounds of the present invention are provided below:

| | IC₅₀ | | | | | | |
|---|---|---|---|---|---|---|---|
| | (nM) | Binding Assay IC₅₀ (nM) | | | Selectivity Binding IC₅₀ | | |
| Compound | hMC4R | hMC1bR | hMC3R | hMC5R | 1R/4R | 3R/4R | 5R/4R |
| A | 44 | 5600 | 7200 | 1600 | 127.27 | 163.6 | 36.36 |
| B | 347 | 7600 | >20000 | 5250 | 21.90 | >57.64 | >15.13 |
| C | 420 | | >20000 | 3900 | | >47.62 | >9.29 |
| D | 182 | | >20000 | 4600 | | >109.89 | >25.27 |
| E | 520 | | >20000 | 6300 | | >38.46 | >12.12 |

### EXAMPLE 2

### cAMP Functional Assay

### To Discriminate Melanocortin Receptor Agonists from Antagonists

Cells (for example, CHO- or L-cells or other eukaryotic cells) expressing a human melanocortin receptor (see e.g. Yang-Y K; Ollmann-M M; Wilson-B D; Dickinson-C; Yamada-T; Barsh-G S; Gantz-I; Mol-Endocrinol. 1997 March; 11(3): 274-80) were dissociated from tissue culture flasks by rinsing with Ca and Mg free phosphate buffered saline (14190-136, Life Technologies, Gaithersburg, Md.) and detached following 5 min incubation at 37° C. with enzyme free dissociation buffer (S-0,4-B, Specialty Media, Lavellette, N.J.). Cells were collected by centrifugation and resuspended in Earle's Balanced Salt Solution (14015-069, Life Technologies, Gaithersburg, Md.) with additions of 10 mM HEPES pH 7.5, 5 mM MgCl₂, 1 mM glutamine and 1 mg/ml bovine serum albumin. Cells were counted and diluted to 1 to 5×10⁶/mL. The phosphodiesterase inhibitor 3-isobutyl-1-methylxanthine was added to cells to 0.6 mM.

Agonist Assay: Test compounds were diluted in dimethylsulfoxide (DMSO) (10⁻⁵ to 10⁻¹⁰ M) and 0.1 volume of compound solution was added to 0.9 volumes of cell suspension; the final DMSO concentration was 1%. After room temperature incubation for 45 min, cells were lysed by incubation at 100° C. for 5 min to release accumulated cAMP.

cAMP was measured in an aliquot of the cell lysate with the Amersham (Arlington Heights, Ill.) cAMP detection assay (RPA556). The amount of cAMP production which resulted from an unknown compound was compared to that amount of cAMP produced in response to alpha-MSH which was defined as a 100% agonist. The EC₅₀ is defined as the compound concentration which results in half maximal stimulation, when compared to its own maximal level of stimulation.

Antagonist assay: Antagonist activity was defined as the ability of a compound to block cAMP production in response to alpha-MSH or other agonists. Solution of test compounds and suspension of receptor containing cells were prepared and mixed as described above; the mixture was incubated for 15 min, and an EC₅₀ dose (approximately 10 nM alpha-MSH) was added to the cells. The assay was terminated at 45 min and cAMP quantitated as above. Percent inhibition was determined by comparing the amount of cAMP produced in the presence to that produced in the absence of test compound.

Melanocortin-4 receptor agonists of use in the present invention are compounds which are potent melanocortin-4 receptor agonists, i.e. compounds with an MC4R functional activity (EC₅₀) less than 90 nM, preferably less than 40 nM, and more preferably less than 15 nM.

Results of cAMP assay (Example 2) and selectivity for representative compounds of the present invention are provided below:

| | EC₅₀ | | | | | | | | |
|---|---|---|---|---|---|---|---|---|---|
| | (nM) | cAMP Assay EC₅₀ (nM) | | | | Selectivity cAMP EC₅₀ | | | |
| Compound | hMC4R | hMC1bR | hMC2R | hMC3R | hMC5R | 1R/4R | 2R/4R | 3R/4R | 5R/4R |
| A | 14 | 1700 | >10000 | 920 | 1400 | 121.43 | >714.2 | 65.71 | 100 |
| B | 88 | 1804 | | 2967 | >5000 | 20.50 | | 33.72 | >56.8 |
| C | 76 | 1100 | | 2625 | >5000 | 14.47 | | 34.54 | >65.7 |
| D | 27 | 580 | | 1500 | 3600 | 21.48 | | 55.56 | 133.3 |
| E | 120 | 1800 | | >5000 | >5000 | 15.00 | | 41.67 | 41.67 |

### EXAMPLE 3

### Maintenance Nicotine Self-Administration Following Treatment of MC4R Agonist

### Compound A

Materials and Methods

Animals: Male Wistar rats were used in all experiments. Body weight was 180-200 g at the start of the experiments. Rats were housed 2-3 per cage with food and water available ad libitum. Lights were on a 12-hour light/dark cycle, with lights on at 0600. All procedures met the guidelines of the National Institutes of Health Guide for the Care and Use of Laboratory Animals.

Operant Nicotine Self-Administration: Nicotine (Sigma Chemical Co., St. Louis, Mo.) was added to saline to achieve a concentration of 0.03 mg/ml. Standard operant chambers (Med Associates, VT) housed in sound-attenuated chambers were used for nicotine self-administration. Syringe pumps delivered nicotine via tygon tubing. The two retractable levers were located 12 cm apart and 4 cm above the floor. Fluid delivery and recording of operant responding were controlled by microcomputer. Rats were trained on a continuous reinforcement fixed-ratio 4 (FR4) schedule to self-administer nicotine (0.03 mg/ml, 0.1 ml per delivery) in 60 minute sessions. Once reliable levels of nicotine-reinforced self-administration were achieved, the effect of Compound A (0, and 20 mg/kg) on maintenance levels of responding was investigated.

Results: Compound A reduced maintenance levels of nicotine self-administration with significant reductions from baseline levels observed at 20 mg/kg (p=0.0001; t=4.9587; df=19). (see FIG. 1). Response rates on the alternate lever were not significantly altered at any dose tested, suggesting the effect of Compound A on response rates was specific to nicotine and not due to a general reduction in operant response rates (See FIG. 2).

These results show that melanocortin 4 agonists, such as Compound A, are useful in reducing nicotine self-administration and consumption.

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the subject being treated for any of the indications for the compounds of the invention indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1-16. (canceled)

17. A method of treating or preventing substance abuse or addiction comprising administering a therapeutically effective amount of a selective melanocortin 4 receptor agonist to a subject in need thereof.

18. The method of claim 17 wherein the melanocortin 4 receptor agonist is orally active.

19. The method of claim 17, wherein the substance is selected from the group consisting of: cocaine, an opiate, an amphetamine, and marijuana.

20. The method of claim 17 wherein the melanocortin 4 receptor agonist is a compound of Formula I or II: 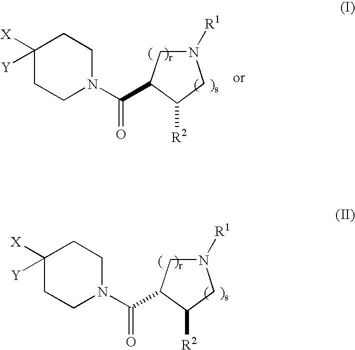
or a pharmaceutically acceptable salt thereof; wherein
X is selected from the group consisting of: C₁₋₈ alkyl, (CH₂)ₙC₃₋₈ cycloalkyl, (CH₂)ₙ-phenyl, (CH₂)ₙ-naphthyl, (CH₂)ₙ-heteroaryl, (CH₂)ₙheterocyclyl, (CH₂)ₙC≡N, (CH₂)ₙCON(R⁵R⁵), (CH₂)ₙCO₂R⁵, (CH₂)ₙCOR⁵, (CH₂)ₙNR⁵C(O)R⁵, (CH₂)ₙNR⁵CO₂R⁵, (CH₂)ₙNR⁵C(O)N(R⁵)₂, (CH₂)ₙNR⁵SO₂R⁵, (CH₂)ₙS(O)ₚR⁵, (CH₂)ₙSO₂N(R⁵)(R⁵), (CH₂)ₙOR⁵, (CH₂)ₙOC(O)R⁵, (CH₂)ₙOC(O)OR⁵, (CH₂)ₙOC(O)N(R⁵)₂, (CH₂)ₙN(R⁵)(R⁵), and (CH₂)ₙNR⁵SO₂N(R⁵)(R⁵); phenyl, naphthyl, and heteroaryl are unsubstituted or substituted with one to three groups independently selected from R⁶; alkyl, cycloalkyl, and heterocyclyl are unsubstituted or substituted with one to three groups independently selected from R⁶ and oxo; and wherein any methylene (CH₂) in X is unsubstituted or substituted with one to two groups independently selected from halogen, hydroxy, and C₁₋₄ alkyl;
Y is selected from the group consisting of: hydrogen, C₁₋₈ alkyl, C₂₋₆ alkenyl, (CH₂)ₙC₃₋₈ cycloalkyl, (CH₂)ₙ-phenyl, (CH₂)ₙ-naphthyl, (CH₂)ₙ-heteroaryl, and (CH₂)ₙ-heterocyclyl; and phenyl, naphthyl, and heteroaryl are unsubstituted or substituted with one to three groups independently selected from R⁶; alkyl, cycloalkyl, and heterocyclyl are optionally substituted with one to three groups independently selected from R⁶ and oxo; and wherein any methylene (CH₂) in Y is unsubstituted or substituted with one to two groups independently selected from halogen, hydroxy, and C₁₋₄ alkyl;
R¹ is selected from the group consisting of: hydrogen, amidino, C₁₋₄ alkyliminoyl, C₁₋₁₀ alkyl, (CH₂)ₙ—C₃₋₇ cycloalkyl, (CH₂)ₙ-phenyl, (CH₂)ₙ-naphthyl, and (CH₂)ₙ-heteroaryl, wherein heteroaryl is selected from the group consisting of (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³; and alkyl and cycloalkyl are unsubstituted or substituted with one to three groups independently selected from R³ and oxo;
R² is selected from the group consisting of: phenyl, naphthyl, and heteroaryl, wherein heteroaryl is selected from the group consisting of: (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³;
each R³ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
each R⁴ is independently selected from the group consisting of: hydrogen, C₁₋₆ alkyl, (CH₂)ₙ phenyl, (CH₂)ₙ-heteroaryl, (CH₂)ₙ-naphthyl, (CH₂)ₙ-heterocyclyl, (CH₂)ₙC₃₋₇ cycloalkyl, and (CH₂)ₙC₃₋₇ bicycloalkyl, wherein alkyl, phenyl, heteroaryl, heterocyclyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl;
each R⁵ is independently selected from the group consisting of: hydrogen, C₁₋₈ alkyl, (CH₂)ₙ-phenyl, (CH₂)ₙ-naphthyl, (CH₂)ₙ-heteroaryl, and (CH₂)ₙC₃₋₇ cycloalkyl; phenyl, naphthyl, and heteroaryl are unsubstituted or substituted with one to three groups independently selected from R³; alkyl and cycloalkyl are unsubstituted or substituted with one to three groups independently selected from R³ and oxo; and wherein any methylene (CH₂) in R⁵ is unsubstituted or substituted with one to two groups independently selected from halogen, hydroxy, and C₁₋₄ alkyl; or two R⁵ groups together with the atom to which they are attached form a 5- to 8-membered mono- or bicyclic ring system optionally containing an additional heteroatom selected from O, S, and NC₁₋₄ alkyl;
each R⁶ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R⁶ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
r is 1 or 2;
s is 0, 1, or 2;
n is 0, 1 or 2; and
p is 0, 1, or 2.

21. A method of facilitating smoking cessation comprising administering a therapeutically effective amount of a selective melanocortin 4 receptor agonist to a subject in need thereof.

22. A method of treating or preventing nicotine addiction comprising administering a therapeutically effective amount of a selective melanocortin 4 receptor agonist to a subject in need of thereof.

23. The method of claim 22 wherein the melanocortin 4 receptor agonist is a compound of Formula I or II: 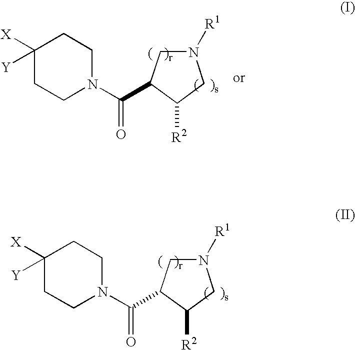
or a pharmaceutically acceptable salt thereof; wherein
X is selected from the group consisting of: C₁₋₈ alkyl, (CH₂)ₙC₃₋₈ cycloalkyl, (CH₂)ₙ-phenyl, (CH₂)ₙ-naphthyl, (CH₂)ₙ-heteroaryl, (CH₂)ₙheterocyclyl, (CH₂)ₙC≡N, (CH₂)ₙCON(R⁵R⁵), (CH₂)ₙCO₂R⁵, (CH₂)ₙCOR⁵, (CH₂)ₙNR⁵C(O)R⁵, (CH₂)ₙNR⁵CO₂R⁵, (CH₂)ₙNR⁵C(O)N(R⁵)₂, (CH₂)ₙNR⁵SO₂R⁵, (CH₂)ₙS(O)ₚR⁵, (CH₂)ₙSO₂N(R⁵)(R⁵), (CH₂)ₙOR⁵, (CH₂)ₙOC(O)R⁵, (CH₂)ₙOC(O)OR⁵, (CH₂)ₙOC(O)N(R⁵)₂, (CH₂)ₙN(R⁵)(R⁵), and (CH₂)ₙNR⁵SO₂N(R⁵)(R⁵); phenyl, naphthyl, and heteroaryl are unsubstituted or substituted with one to three groups independently selected from R⁶; alkyl, cycloalkyl, and heterocyclyl are unsubstituted or substituted with one to three groups independently selected from R⁶ and oxo; and wherein any methylene (CH₂) in X is unsubstituted or substituted with one to two groups independently selected from halogen, hydroxy, and C₁₋₄ alkyl;
Y is selected from the group consisting of: hydrogen, C₁₋₈ alkyl, C₂₋₆ alkenyl, (CH₂)ₙC₃₋₈ cycloalkyl, (CH₂)ₙ-phenyl, (CH₂)ₙ-naphthyl, (CH₂)ₙ-heteroaryl, and (CH₂)ₙ-heterocyclyl; and phenyl, naphthyl, and heteroaryl are unsubstituted or substituted with one to three groups independently selected from R⁶; alkyl, cycloalkyl, and heterocyclyl are optionally substituted with one to three groups independently selected from R⁶ and oxo; and wherein any methylene (CH₂) in Y is unsubstituted or substituted with one to two groups independently selected from halogen, hydroxy, and C₁₋₄ alkyl;
R¹ is selected from the group consisting of: hydrogen, amidino, C₁₋₄ alkyliminoyl, C₁₋₁₀ alkyl, (CH₂)ₙ—C₃₋₇ cycloalkyl, (CH₂)ₙ-phenyl, (CH₂)ₙ-naphthyl, and (CH₂)ₙ-heteroaryl, wherein heteroaryl is selected from the group consisting of (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³; and alkyl and cycloalkyl are unsubstituted or substituted with one to three groups independently selected from R³ and oxo;
R² is selected from the group consisting of: phenyl, naphthyl, and heteroaryl, wherein heteroaryl is selected from the group consisting of: (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³;
each R³ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
each R⁴ is independently selected from the group consisting of: hydrogen, C₁₋₆ alkyl, (CH₂)ₙ phenyl, (CH₂)ₙ-heteroaryl, (CH₂)ₙ-naphthyl, (CH₂)ₙ-heterocyclyl, (CH₂)ₙC₃₋₇ cycloalkyl, and (CH₂)ₙC₃₋₇ bicycloalkyl, wherein alkyl, phenyl, heteroaryl, heterocyclyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl;
each R⁵ is independently selected from the group consisting of: hydrogen, C₁₋₈ alkyl, (CH₂)ₙ-phenyl, (CH₂)ₙ-naphthyl, (CH₂)ₙ-heteroaryl, and (CH₂)ₙC₃₋₇ cycloalkyl; phenyl, naphthyl, and heteroaryl are unsubstituted or substituted with one to three groups independently selected from R³; alkyl and cycloalkyl are unsubstituted or substituted with one to three groups independently selected from R³ and oxo; and wherein any methylene (CH₂) in R⁵ is unsubstituted or substituted with one to two groups independently selected from halogen, hydroxy, and C₁₋₄ alkyl; or two R⁵ groups together with the atom to which they are attached form a 5- to 8-membered mono- or bicyclic ring system optionally containing an additional heteroatom selected from O, S, and NC₁₋₄ alkyl;
each R⁶ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC_N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R⁶ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
r is 1 or 2;
s is 0, 1, or 2;
n is 0, 1 or 2; and
p is 0, 1, or 2.

24. A method of reducing nicotine consumption comprising administration of a therapeutically effective amount of a selective melanocortin 4 receptor agonist to a subject in need thereof.

25. The method of claim 24 wherein the melanocortin 4 receptor agonist has a selective functional activity characterized by an EC₅₀ at least 65-fold lower for the human melanocortin 4 receptor than for the human melanocortin 1 receptor, the melanocortin 2 receptor, the human melanocortin 3 receptor and the human melanocortin 5 receptor.

26. The method of claim 24 wherein the functional activity of the melanocortin 4 receptor agonist is characterized by an EC₅₀ at least 120-fold lower for the human melanocortin 4 receptor than for the human melanocortin 1 receptor.

27. The method of claim 24 wherein the functional activity of the melanocortin 4 receptor agonist is characterized by an EC₅₀ at least 700-fold lower for the human melanocortin 4 receptor than for the human melanocortin 2 receptor.

28. The method of claim 24 wherein the functional activity of the melanocortin 4 receptor agonist is characterized by an EC₅₀ at least 90-fold lower for the human melanocortin 4 receptor than for the human melanocortin 5 receptor.

29. The method of claim 24 wherein the melanocortin 4 receptor agonist has an IC₅₀ value of less than 45 nM at the human melanocortin 4 receptor.

30. The method of claim 24 wherein the melanocortin 4 receptor agonist is a compound of Formula I or II: 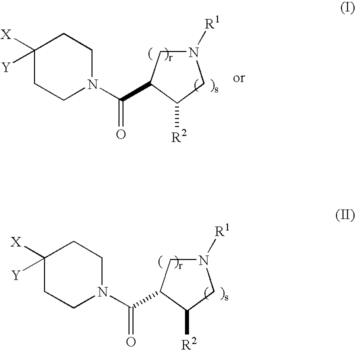
or a pharmaceutically acceptable salt thereof, wherein
X is selected from the group consisting of: C₁₋₈ alkyl, (CH₂)ₙC₃₋₈ cycloalkyl, (CH₂)ₙ-phenyl, (CH₂)ₙ-naphthyl, (CH₂)ₙ-heteroaryl, (CH₂)ₙheterocyclyl, (CH₂)ₙC≡N, (CH₂)ₙCON(R⁵R⁵), (CH₂)ₙCO₂R⁵, (CH₂)ₙCOR⁵, (CH₂)ₙNR⁵C(O)R⁵, (CH₂)ₙNR⁵CO₂R⁵, (CH₂)ₙNR⁵C(O)N(R⁵)₂, (CH₂)ₙNR⁵SO₂R⁵ (CH₂)ₙS(O)ₚR⁵, (CH₂)ₙSO₂N(R⁵)(R⁵), (CH₂)ₙOR⁵, (CH₂)ₙOC(O)R⁵, (CH₂)ₙOC(O)OR⁵, (CH₂)ₙOC(O)N(R⁵)₂, (CH₂)ₙN(R⁵)(R⁵), and (CH₂)ₙNR⁵SO₂N(R⁵)(R⁵); phenyl, naphthyl, and heteroaryl are unsubstituted or substituted with one to three groups independently selected from R⁶; alkyl, cycloalkyl, and heterocyclyl are unsubstituted or substituted with one to three groups independently selected from R⁶ and oxo; and wherein any methylene (CH₂) in X is unsubstituted or substituted with one to two groups independently selected from halogen, hydroxy, and C₁₋₄ alkyl;
Y is selected from the group consisting of: hydrogen, C₁₋₈ alkyl, C₂₋₆ alkenyl, (CH₂)ₙC₃₋₈ cycloalkyl, (CH₂)ₙ-phenyl, (CH₂)ₙ-naphthyl, (CH₂)ₙ-heteroaryl, and (CH₂)ₙ-heterocyclyl; and phenyl, naphthyl, and heteroaryl are unsubstituted or substituted with one to three groups independently selected from R⁶; alkyl, cycloalkyl, and heterocyclyl are optionally substituted with one to three groups independently selected from R⁶ and oxo; and wherein any methylene (CH₂) in Y is unsubstituted or substituted with one to two groups independently selected from halogen, hydroxy, and C₁₋₄ alkyl;
R¹ is selected from the group consisting of: hydrogen, amidino, C₁₋₄ alkyliminoyl, C₁₋₁₀ alkyl, (CH₂)ₙ—C₃₋₇ cycloalkyl, (CH₂)ₙ-phenyl, (CH₂)ₙ-naphthyl, and (CH₂)ₙ-heteroaryl, wherein heteroaryl is selected from the group consisting of (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³; and alkyl and cycloalkyl are unsubstituted or substituted with one to three groups independently selected from R³ and oxo;
R² is selected from the group consisting of: phenyl, naphthyl, and heteroaryl, wherein heteroaryl is selected from the group consisting of: (1) pyridinyl, (2) furyl, (3) thienyl, (4) pyrrolyl, (5) oxazolyl, (6) thiazolyl, (7) imidazolyl, (8) pyrazolyl, (9) isoxazolyl, (10) isothiazolyl, (11) pyrimidinyl, (12) pyrazinyl, (13) pyridazinyl, (14) quinolyl, (15) isoquinolyl, (16) benzimidazolyl, (17) benzofuryl, (18) benzothienyl, (19) indolyl, (20) benzthiazolyl, and (21) benzoxazolyl; in which phenyl, naphthyl, and heteroaryl are unsubstituted or substituted with one to three groups independently selected from R³; each R³ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R³ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
each R⁴ is independently selected from the group consisting of: hydrogen, C₁₋₆ alkyl, (CH₂)ₙ phenyl, (CH₂)ₙ-heteroaryl, (CH₂)ₙ-naphthyl, (CH₂)ₙ-heterocyclyl, (CH₂)ₙC₃₋₇ cycloalkyl, and (CH₂)ₙC₃₋₇ bicycloalkyl, wherein alkyl, phenyl, heteroaryl, heterocyclyl, and cycloalkyl are unsubstituted or substituted with one to three groups independently selected from halogen, C₁₋₄ alkyl, hydroxy, and C₁₋₄ alkoxy; or two R⁴ groups together with the atom to which they are attached form a 4- to 8-membered mono- or bicyclic ring system optionally containing an additional heteroatom selected from O, S, and NC₁₋₄ alkyl;
each R⁵ is independently selected from the group consisting of: hydrogen, C₁₋₈ alkyl, (CH₂)ₙ-phenyl, (CH₂)ₙ-naphthyl, (CH₂)ₙ-heteroaryl, and (CH₂)ₙC₃₋₇ cycloalkyl; phenyl, naphthyl, and heteroaryl are unsubstituted or substituted with one to three groups independently selected from R³; alkyl and cycloalkyl are unsubstituted or substituted with one to three groups independently selected from R³ and oxo; and wherein any methylene (CH₂) in R⁵ is unsubstituted or substituted with one to two groups independently selected from halogen, hydroxy, and C₁₋₄ alkyl; or two R⁵ groups together with the atom to which they are attached form a 5- to 8-membered mono- or bicyclic ring system optionally containing an additional heteroatom selected from O, S, and NC₁₋₄ alkyl;
each R⁶ is independently selected from the group consisting of: C₁₋₆ alkyl, (CH₂)ₙ-phenyl, (CH₂)ₙ-naphthyl, (CH₂)ₙ-heteroaryl, (CH₂)ₙ-heterocyclyl, (CH₂)ₙC₃₋₇ cycloalkyl, halogen, OR⁴, (CH₂)ₙN(R⁴)₂, (CH₂)ₙC≡N, (CH₂)ₙCO₂R⁴, NO₂, (CH₂)ₙNR⁴SO₂R⁴, (CH₂)ₙSO₂N(R⁴)₂, (CH₂)ₙS(O)ₚR⁴, (CH₂)ₙNR⁴C(O)N(R⁴)₂, (CH₂)ₙC(O)N(R⁴)₂, (CH₂)ₙNR⁴C(O)R⁴, (CH₂)ₙNR⁴CO₂R⁴, (CH₂)ₙNR⁴C(O)-heteroaryl, (CH₂)ₙC(O)NR⁴N(R⁴)₂, (CH₂)ₙC(O)NR⁴NR⁴C(O)R⁴, O(CH₂)ₙC(O)N(R⁴)₂, CF₃, CH₂CF₃, OCF₃, and OCH₂CF₃; phenyl, naphthyl, heteroaryl, cycloalkyl, and heterocyclyl are unsubstituted or substituted with one to three substituents independently selected from halogen, hydroxy, oxo, C₁₋₄ alkyl, trifluoromethyl, and C₁₋₄ alkoxy; and wherein any methylene (CH₂) carbon atom in R⁶ is unsubstituted or substituted with one to two groups independently selected from halogen, hydroxy, and C₁₋₄ alkyl; or two substituents when on the same methylene (CH₂) group are taken together with the carbon atom to which they are attached to form a cyclopropyl group;
r is 1 or 2;
s is 0, 1, or 2;
n is 0, 1 or 2; and
p is 0, 1, or 2.

31. The method of claim 30 wherein X is phenyl substituted with two groups independently selected from R³, Y is hydrogen, and R¹ is tert-butyl, and R² is phenyl substituted with two groups independently selected from R³, or a pharmaceutically acceptable salt thereof.

32. The method of claim 30 wherein the melanocortin 4 receptor agonist of Formula I is 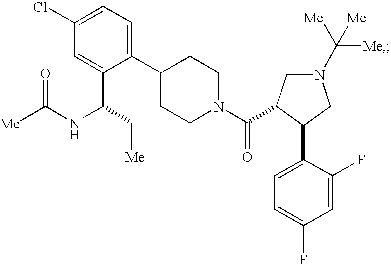
or a pharmaceutically acceptable salt thereof.
